(19) United States
(12) Patent Application Publication
(10) Pub. No.: US 2023/0146939 A1
(43) Pub. Date: May 11, 2023

(54) TREATMENT FOR HUMAN CORONAVIRUS INFECTION

(71) Applicant: THE ADMINISTRATORS OF THE TULANE EDUCATIONAL FUND, New Orleans, LA (US)

(72) Inventor: Gregory Jaye Bix, New Orleans, LA (US)

(21) Appl. No.: 18/010,731

(22) PCT Filed: Jun. 15, 2021
(86) PCT No.: PCT/US21/37486

§ 371 Date: Dec. 15, 2022

Related U.S. Application Data
(60) Provisional application No. 63/039,277, filed on Jun. 15, 2020.

(51) Int. Cl. A61K 31/4178; A61P 31/14

(52) U.S. Cl. CPC A61K 31/4178; A61P 31/14

(57) ABSTRACT

Treating a human coronavirus (hCoV) infection includes administering an effective amount of an inhibitor of an α5- or αv-containing integrin to a subject in need thereof. The integrin inhibitor can be ATN-161. The hCoV can be SARS-CoV or SARS-CoV-2.

### RELATED APPLICATIONS

This application claims priority from U.S. Provisional Application Ser. No. 63/039,277 filed Jun. 15, 2020, the entire disclosure of which is incorporated herein by this reference.

### TECHNICAL FIELD

The presently-disclosed subject matter generally relates to treating coronavirus infection. In particular, certain embodiments of the presently-disclosed subject matter relate to methods of using an inhibitor of an α5- or αv-containing integrin to treat, ameliorate, or prevent a human coronavirus (hCoV) infection or disease.

### INTRODUCTION

Severe acute respiratory syndrome coronavirus 2 (SARS-CoV-2) is the causative agent of severe acute respiratory syndrome 2, which can lead to coronavirus disease 2019 (COVID-19). SARS-CoV-2 is a highly transmissible respiratory pathogen in which an estimated 14% of all patients will develop serious conditions, with a subsequent mortality rate of 1.4-3.4% [1-3]. SARS-CoV-2 is an enveloped virus, encased with a lipid membrane containing spike proteins. The viral spike proteins bind to angiotensin-converting enzyme 2 (ACE2) receptors on target host cells to facilitate its entry into and replication by the host cells[9].

Several non-pharmacological interventions have been implemented to slow down the spread of SARS-CoV-2[4-7]. However, there remains a need for therapies to treat COVID-19. Potential strategies could employ targets for disrupting aspects of the viral replication process, including SARS-CoV-2 host cell entry [8]. The virus infects the cells after the proteolytic cleavage of the spike protein by the transmembrane serine protease 2 (TMPRSS2) or Cathepsin B or L or FURIN is required for spike protein priming and virus infection [10].

Integrins are family of cell adhesion receptors composed of non-covalently linked α and β subunits that recognize and bind to extracellular matrix (ECM) proteins and mediate cell survival, proliferation, differentiation, and migration [15-18]. Integrin dimers are expressed in most cells, including endothelial cells, and epithelial cells in the respiratory tract [19], and are known to be involved in the infectious etiology of other viruses such as human cytomegalovirus [20], Epstein—Barr virus [21], rotavirus [22], Kaposi's sarcoma-associated virus (HHV-8) [23] and Ebola [24]. The β1 family of integrins are closely associated with ACE2 [25]. Integrins could play a role in SARS-CoV-2 host cell entry and infection.

A novel mutation (K403R) has been identified in the spike protein that does not exist in other strains of the coronavirus, creating an integrin binding motif arginine-glycine-aspartate (RGD) sequence[2]. Therefore, the new RGD motif in SARS-CoV-2 could increase the binding potency of ACE2-positive target cells in association with β1 integrins as well as potentially facilitating infection of ACE2-negative cells [2, 11-14]. This could help explain the faster and aggressive spread of virus as compared to SARS-CoV-1, which belongs to the same family of beta coronaviruses [26].

However, further developments have complicated the understanding of the potential role of integrins in this context. Indeed, research has recently suggested that the SARS-CoV-2 spike protein does not bind to integrins via the RGD motif [76]. Meanwhile, as new variants of the virus emerge, mutations are occurring that could result in the formation of more stable spike protein-ACE2 complexes, which could result in increased infectivity of the variants. [77]

Accordingly, there remains a need in the art for effective therapies to reduce and treat infection by a coronavirus.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method of treating a human coronavirus (hCoV) infection, which involves comprising administering an effective amount of an integrin inhibitor of an α5- or αv-containing integrin to a subject in need thereof. The presently-disclosed subject matter includes use of an integrin inhibitor of an α5- or αv-containing integrin in the manufacture of a medicament for the treatment of a human coronavirus (hCoV) infection.

In some embodiments of the presently-disclosed subject matter the integrin inhibitor is an α5β1 integrin inhibitor. In some embodiments, the integrin inhibitor comprises an ATN-161. In some embodiments, the ATN-161 is provided as pharmaceutically-acceptable salt. In some embodiments, the ATN-161 is provided as a trifluoroacetate salt. In some embodiments, the ATN-161 is adapted for administration intranasally, pulmonarily, orally, or parenterally.

In some embodiments of the presently-disclosed subject matter the hCoV is severe acute respiratory syndrome (SARS) CoV or SARS-CoV-2. In some embodiments, the hCoV includes a spike protein that binds to an angiotensin-converting enzyme 2 (ACE2) of a target host cell. In some embodiments, the hCoV binds an α5- or αv-containing integrin. In some embodiments, the integrin is α5β1 integrin.

In some embodiments of the presently-disclosed subject matter the subject has been exposed to or is at risk of being exposed to the hCoV. In some embodiments, the integrin inhibitor is administered prophylactically to the subject. In some embodiments, the method involves administering the integrin inhibitor to the subject at least three times a week. In some embodiments, the method involves administering the integrin inhibitor to the subject for at least 1 week. In some embodiments, the method involves administering the integrin inhibitor to the subject for at least 10 days. In some embodiments, the method involves administering the integrin inhibitor to the subject until the risk of being exposed to hCoV is reduced.

In some embodiments of the presently-disclosed subject matter the subject has been infected with the hCoV. In some embodiments, the subject has tested positive for the hCoV. In some embodiments, the subject has at least one symptom of SARS or COVID. In some embodiments, the method involves administering the integrin inhibitor to the subject at least three times a week. In some embodiments, the method involves administering the integrin inhibitor to the subject for at least 1 week. In some embodiments, the method involves administering the integrin inhibitor to the subject for at least 10 days. In some embodiments, the method involves administering the integrin inhibitor to the subject until the at least one symptom has resolved.

In some embodiments of the presently-disclosed subject matter viral activity is reduced in the subject. In some embodiments of the presently-disclosed subject matter there is protection against detrimental histopathological changes.

The presently-disclosed subject matter includes a pharmaceutical composition for use in the treatment of a human coronavirus (hCoV) infection, comprising an integrin inhibitor of an α5- or αv-containing integrin and a pharmaceutically-acceptable carrier. In some embodiments, the integrin inhibitor is an α5β1 integrin inhibitor. In some embodiments, the integrin inhibitor comprises an ATN-161. In some embodiments, the ATN-161 is provided as a pharmaceutically-acceptable salt. In some embodiments, the ATN-161 is provided as a trifluoroacetate salt. In some embodiments, the ATN-161 is adapted for administration intranasally, pulmonarily, orally, or parenterally.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 is a schematic illustration showing ATN-161 blocking (Xs) SARS-CoV-2 spike protein (S_{SARS-CoV-2}) binding and subsequent host cell SARS-CoV-2 uptake primarily by blocking its interactions with α5β1 integrin and ACE2, the latter both indirectly via this integrin blockade, and directly.

FIG. 2A shows inhibition of binding of α5β1 to SARS-CoV-2 trimeric spike protein by ATN-161.

FIG. 2B shows inhibition of binding of α5β1 to ACE2 by ATN-161.

FIG. 3A shows inhibition of binding of SARS-CoV-2 trimeric spike protein to human ACE2 by ATN-161.

FIG. 3B shows inhibition of binding of SARS-CoV-2 monomeric spike protein to human ACE2 by ATN-161.

FIG. 4 shows the viral load of SARS-CoV-2 in VeroE6 cells with and without treatment using ATN-161.

FIG. 5 shows cell viability via luminescence-based CellTiterGlo 24 hours post infection with ATN-161 treatment.

FIG. 6 depicts ATN-161 reduction of SARS-CoV-2 infectivity in Vero E6 Cells, as shown by a photomicrograph of Vero E6 cells infected with SARS-CoV-2 for 24 hours+/−ATN-161.

FIGS. 7A-7D illustrate that ATN-161 reduces genomic-N and sub-genomic N (Sgm-N) viral load in SARS-CoV-2 infected K18-hACE2 mice lung tissue. FIG. 7A includes a schematic overview of experimental timeline for K18-hACE2 mice. FIG. 7B shows the resulting expression in lungs of K18-hACE2 mice, FIG. 7C shows the resulting viral genomic-N(Total-N) mRNA, and FIG. 7D shows the resulting sub-genomic-N mRNA (sgm-N).

FIGS. 8A-8C illustrate that ATN-161 reduces the expression of virus in SARS-CoV-2 infected K18-hACE2 mice lung tissue. FIG. 8A includes four panels providing representative images of immunohistochemistry staining for SARS-CoV-2 in lung tissue from mice that received: (i) saline/ATN-161, (ii) SARS-CoV-2+vehicle (iii) responders with SARS-CoV-2+ATN-161 administration, and (iv) Non-responders with SARS-CoV-2+ATN-161 administration. Scale bars are 50 μm. FIGS. 8B and 8C are bar graphs that present quantified results, where FIG. 8B pools the responders and non-responders (fourth bar), and FIG. 8C presents the responders and non-responders separately (fourth and fifth bars). Data are presented as mean±SEM; P values represent saline vs SARS-CoV-2+vehicle and SARS-CoV-2+vehicle vs SARS-CoV-2+ATN-161 pooled, SARS-CoV-2+ATN-161 responders, and SARS-CoV-2+ATN-161 non-responders

FIGS. 9A-9D include representative images from histopathological analysis of SARS-CoV-2 infected K18-hACE2 mice lung tissue, conducted using hematoxylin and eosin staining of lung sections. Each figure includes two panels providing images at different scales, where the scale bars are 500 μm in each panel (i) and the scale bars are 200 μm in each panel (ii). FIG. 9A includes representative images from mice administered saline and ATN-161. FIG. 9B includes representative images from mice administered SARS-CoV-2 and vehicle. FIG. 9C includes representative images from mice administered SARS-CoV-2+ATN-161, which mice were responders. FIG. 9D includes representative images from mice administered SARS-CoV-2+ATN-161, which mice were non-responders.

FIGS. 10A-10D include representative images from histopathological analysis of SARS-CoV-2 infected K18-hACE2 mice lung tissue, conducted using Masson's Trichrome-stained lung sections. Each figure includes two panels providing images at different scales, where the scale bars are 500 μm in each panel (i) and the scale bars are 200 μm in each panel (ii). FIG. 10A includes representative images from mice administered saline and ATN-161. FIG. 10B includes representative images from mice administered SARS-CoV-2 and vehicle. FIG. 10C includes representative images from mice administered SARS-CoV-2+ATN-161, which mice were responders. FIG. 20D includes representative images from mice administered SARS-CoV-2+ATN-161, which mice were non-responders.

FIGS. 11A-11D illustrate that induced expression of integrin α5, and integrin αv in the lungs of SARS-CoV-2 infected K18-hACE2 mice and is inhibited by ATN-161 treatment. FIGS. 11A-11B include integrin α5 mRNA expression in lung tissue from mice receiving saline, ANT-161, SARS-CoV-2 (TCID50=2×10⁵/0.05 mL), or SARS-CoV-2+ATN-161. FIG. 11A pools the responders and non-responders (fourth bar), and FIG. 11B presents the responders and non-responders separately (fourth and fifth bars). FIGS. 11C-11D include integrin αv mRNA expression in lung tissue from mice receiving saline, ANT-161, SARS-CoV-2 (TCID50=2×10⁵/0.05 mL), or SARS-CoV-2+ATN-161. FIG. 11C pools the responders and non-responders (fourth bar), and FIG. 11D presents the responders and non-responders separately (fourth and fifth bars).

FIGS. 12A-12C present lung outcomes following SARS-CoV-2 with and without ATN-161 pre-treatment. Tissues collected from treated mice were analyzed using RT-PCR for genomic-N, with results shown in FIG. 12A, and Sub-genomic N (Sgm-N), with results shown in FIG. 12B. Integrin alpha 5 mRNA express in lungs was also analyzed, with results shown in FIG. 12C.

FIGS. 13A and 13B show the effects of ATN-161 on SARS-CoV-2 spike variants, and α5b1 Binding. Enzyme-linked immunosorbent assay data indicates that ATN-161 alters binding of α5β1 to Spike Variants, including the South Africa Variant (NR-54005), with results shown in FIG. 13A, and United Kingdom Variant (NR-54004), with results shown in FIG. 13B.

### BRIEF DESCRIPTION OF THE SEQUENCE LISTING

This application contains a sequence listing submitted in accordance with 37 C.F.R. 1.821, named Bix-TM-655—PCT Application Sequence Listing ST25.txt, created on Jun. 15, 2021, having a size of 5 KB, which is incorporated herein by this reference.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods and compositions for use in treating a human coronavirus (hCoV) infection. Coronaviruses are enveloped viruses, encased with a lipid membrane containing spike protein, which is sometimes referred to as S protein. Spike protein is highly conserved among all human coronaviruses and is involved in recognition of receptors on a target host cell to facilitate entry into and replication by the target host cell [78]. As used herein, the term “human coronavirus” or “hCoV” refers to a coronavirus that is capable of infecting a human subject. Accordingly, excluded from this term are coronaviruses that are only capable of infecting a non-human subject. Examples of human coronavirus include, but are not limited to: alpha coronaviruses such as 229E and NL63, beta coronaviruses such as OC43 and HKU1, Middle East Respiratory Syndrome (MERS) coronavirus (CoV), severe acute respiratory syndrome (SARS) CoV, SARS-CoV-2, and variants thereof.

The presently-disclosed subject matter is useful in the treatment of hCoV infection because methods and compositions according to the presently-disclosed subject matter can prevent or reduce hCoV viral activity in a human subject who has been exposed to, who is at risk of being exposed to, or who has been infected with hCoV.

As will be appreciated by one of ordinary skill in the art upon study of the present document, the terms “treat,” “treatment,” and the like refer to the medical management of a subject with the intent to cure, ameliorate, stabilize, or prevent an infection or disease. This term includes treatment that is directed toward: direct treatment directed to the improvement of an infection state or disease state; causal treatment directed to the removal of the cause of an infection or disease; palliative treatment directed to the relief or amelioration of symptoms of the infection or disease, preventative or prophylactic treatment directed to minimizing or partially or completely inhibiting an infection or development of an associated disease; and supportive treatment that is directed supplementing another specific therapy directed toward treatment of an infection or disease.

With regard to preventative or prophylactic treatment, terms such as “prevent,” “block,” “inhibit,” and “protect against” as used herein are not absolute, rather, they refers to the characteristic of elimination and are also inclusive of reduction. Thus, such terms do not imply complete elimination or a particular degree of reduction of an infection, disease, or characteristic or symptom thereof. Instead, the terms can be used to refer to z result that id less than s 100% reduction relative to a control. Thus, in some embodiments, the terms can be used to refer to a reduction in an infection, disease, or characteristic or symptom thereof by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% relative to a control that has not received the treatment.

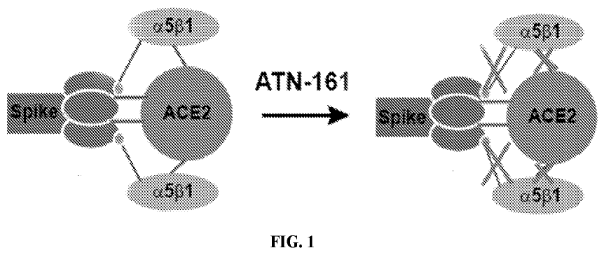

The presently-disclosed subject matter is based, at least in part, on the unique discoveries disclosed herein. The β1 family of integrins are associated with ACE2 [25]. Integrins α5β1 and αvβ1 are members of the β1 family of integrins, and fibrinogen is a natural ligand of both α5β1 and αvβ1. Increased fibrinogen has been found to be associated with lung conditions [80]. As disclosed herein, there is increased expression of integrin α5 and integrin αv in the lungs of SARS-CoV-2 infected animals. See Examples 3 and 4. As also disclosed herein, α5β1 integrin binds ACE-2 and also binds SARS-CoV-2 spike protein. See Example 1. Spike protein is highly conserved among human coronaviruses [78]. The binding of α5β1 to both ACE-2 and spike protein facilitates the direct binding of ACE-2 and the spike protein. See FIG. 1, left panel; see also Example 1. As disclosed herein, ATN-161 was found to disrupt binding of α5β1 to both ACE-2 and spike protein. See Example 1. ATN-161 is an α5β1 integrin inhibitor, and can also interacts with other β1 integrins and αv integrins. As disclosed herein, ATN-161 was also found to disrupt binding between ACE-2 and the SARS-CoV-2 trimeric spike protein. See Example 1.

These findings support use of an integrin inhibitor of an α5- or αv-containing integrin for use in treating hCoV infection, including prophylactic treatment by preventing viral entry due to the inhibition of binding between ACE-2 and spike protein-ACE-2 that facilitates entry into and replication by a target host cell.

The findings disclosed herein also support therapeutic use in a subject who has been infected with hCoV. As disclosed herein, animals infected with SARS-CoV2 have increased levels of expression of integrin α5 and integrin ay. See Examples 3 and 4. This creates an environment in which there is a higher risk for direct binding between ACE-2 and the spike protein, and subsequent viral entry into host cells. In this context, and as noted above, both α5- and αv-integrins bind fibronectin and fibronectin is known to be upregulated in certain lung condition. In this regard, administration of an α5- or αv-integrin inhibitor can have an added benefit of disrupting binding of the natural ligand, fibronectin, in the context of a lung condition in which the α5- or αv-integrins as well as their natural ligand are upregulated. SARS is an example a lung condition that is caused by hCoVs.

Additionally, as disclosed herein, ATN-161 inhibits the increased express of integrin α5 and integrin ay. See Example 3. Therefore, ATN-161 mitigates the increased risk of integrin-facilitated binding between ACE-2 and the spike protein by inhibiting the increased integrin expression, thereby reducing the spread of infection in an animal that has been infected with hCoV. Furthermore, in an animal that has been infected with hCoV, ATN-161 will protects against infection because it inhibits the spike protein-ACE-2 binding of additional target cells within the infected subject. As also disclosed herein, in cells infected with SARS-CoV-2, ATN-161 decreases viral load in cells, increases cell viability, and reduces cytopathic effect. See Example 2. Additionally, in animals infected with SARS-CoV-2, ATN-161 reduces lung viral load, reduces viral replication in lung, and improves lung histology. See Examples 3 and 4.

These findings support use of an integrin inhibitor of an α5- or αv-containing integrin for use in treating hCoV infection, including protection against detrimental histopathological changes and reduction of viral activity in the subject. Such viral activity can include, for example, viral entry, viral load, viral replication, and viral infection.

The presently-disclosed subject matter includes a method of treating a human coronavirus (hCoV) infection that involves administering an effective amount of an integrin inhibitor of an α5- or αv-containing integrin to a subject in need thereof. The presently-disclosed subject matter includes use of an integrin inhibitor of an α5- or αv-containing integrin in the manufacture of a medicament for the treatment of a human coronavirus (hCoV) infection. The presently-disclosed subject matter includes a composition for use in the treatment of a human coronavirus (hCoV) infection, comprising an integrin inhibitor of an α5- or αv-containing integrin and a pharmaceutically-acceptable carrier. In some embodiments of the presently-disclosed subject matter the integrin inhibitor that is employed is an α5β1 integrin inhibitor.

Examples of inhibitors of α5- or αv-containing integrin include, but are not limited to: vitaxin, cilengitide, CWHM-12, Cyclo(-RGDfK), iRGD peptide, αvβ1 integrin-IN-1 (Compound C8), HSDVHK-NH2, MK-0429, Echistatin, CRRETAWAC (SEQ ID NO: 8) inhibitor peptide, and ATN-161.

In some embodiments of the presently-disclosed subject matter the integrin inhibitor that is employed is ATN-161. ATN-161 is a non-RGD peptide with the amino acid sequence PHSCN (SEQ ID NO: 1) that has been developed to inhibit α5β1 integrin and has been tested in experimental models and clinical trials as a therapeutic for conditions such as cancer and stroke. Among other things, ATN-161 can be safely administered by various routes, and has a relatively short serum half-life (e.g., 3-5 h for some subjects i.v. and closer to about 1 day for other subjects and administration routes) with high tissue distribution. ATN-161, in addition to noncovalent interaction with the α5 integrin subunit, can have a covalent interaction with the β1 and β3 subunit of α5β1 and αvβ3 integrin. ATN-161 is sometimes referred to as Ac-PHSCN-NH2 (CAS No. 262438-43-7). In some cases ATN-161 is provided in a modified form. Ac-PhScN-NH2 is a modified version of ATN-161 in which the covalent interaction is eliminated by replacing the H and C with D-stereoisomers. Ac-PhScN-NH₂ specifically and noncovalently interacts with the α5 subunit of α5β1 integrin and is between 27,000 fold and 379,000 fold more potent and specifically inhibits activated α5131 integrin mediated processes. As used herein, the term “ATN-161” refers to al known versions of ATN-161 including Ac-PHSCN-NH₂ and Ac-PhScN-NH₂. In some embodiments, ATN-161 can be provided as pharmaceutically-acceptable salt. In some embodiments, ATN-161 is provided as a trifluoroacetate (TFA) salt. In some embodiments, the ATN-161 is adapted for administration intranasally, pulmonarily, orally, or parenterally.

In accordance with the presently-disclosed subject matter, the term “administering” refers to any method of providing the integrin inhibitor to a subject. Such methods are well known to those skilled in the art and include, but are not limited to, oral administration, transdermal administration, nasal administration, intracerebral administration, and administration by injection, which itself can include intravenous administration, intra-arterial administration, intramuscular administration, subcutaneous administration, intravitreous administration, intracameral (into anterior chamber) administration, and the like. Administration can be continuous or intermittent. In various aspects, a preparation can be administered therapeutically; that is, administered to treat an existing disease or condition (e.g., ischemia, infarction, etc.). In other instances a preparation is administered prophylactically; that is, administered to prevent or treat a disease or condition that may otherwise develop.

As used herein, the terms “effective amount” refers to a dosage sufficient to provide treatment. The exact amount that is required will vary from subject to subject, depending on the species, age, and general condition of the subject, the particular carrier or adjuvant being used, mode of administration, and the like. As such, the effective amount will vary based on the particular circumstances, and an appropriate effective amount can be determined in a particular case by one of ordinary skill in the art using only routine experimentation.

In some instances an effective amount is determined relative to the weight of a subject, and can be selected from dosages of about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, and 50 mg/kg.

The term “subject” is used herein to refer to a target of administration and includes an animal capable of being infected by a human coronavirus (hCoV). In some embodiments the subject is a human.

As described herein, the presently-disclosed subject matter is directed to treatment of a human coronavirus (hCoV) infection. In some embodiments, the hCoV includes a spike protein that binds to an angiotensin-converting enzyme 2 (ACE2) of a target host cell. In some embodiments, the hCoV binds an α5- or αv-containing integrin. In some embodiments, the hCoV binds α5β1 integrin. In some embodiments, the hCoV is selected from the group consisting of alpha coronaviruses including 229E and NL63, beta coronaviruses including OC43 and HKU1, Middle East Respiratory Syndrome (MERS) coronavirus (CoV), severe acute respiratory syndrome (SARS) CoV, SARS-CoV-2, and variants thereof. In some embodiments, the hCoV is selected from SARS-CoV and SARS-CoV-2.

In some embodiments of the presently-disclosed subject matter, the subject has been exposed to or is at risk of being exposed to the hCoV. In this regard, the integrin inhibitor can be administered prophylactically to the subject. A treatment regimen can be designed in view of a particular situation, in accordance with recommendations from one of ordinary skill in the art. In some embodiments, the integrin inhibitor is administered multiple times over a treatment period. For example, in some embodiments, the integrin inhibitor is administered to the subject about 3 times a week to about 21 times a week. In this regard, in some embodiments, administration occurs on 3, 4, 5, 6, or 7 days of a week, and in other embodiments, administration occurs once, twice, or three times each day of a week. Furthermore, in some embodiments the treatment period could be one week, two weeks, three weeks, 4 weeks, or more. In some embodiments, the treatment period could be 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days. In some embodiments, the treatment period is ongoing until the risk of being exposed to hCoV is reduced or substantially eliminated.

In some embodiments of the presently-disclosed subject matter, the subject has been infected with the hCoV. Infection status can be determined, for example, using a test to determine the presence of the hCoV in a sample obtained from the subject. Various tests are currently available, including those that test for presence of an antigen or for presence of a nucleic acid associated with the CoV. In some embodiments, a positive test can be used to identify whether the subject has been infected.

In some embodiments, infection status can be determined based on the presence of one or more symptoms of a disease caused by the hCoV (e.g., SARS or COVID). Symptoms of SARS and of COVID will be recognized by the skilled artisan. Examples of such symptoms include, but are not limited to, fever (temperature greater than about 100.4° F.), chills, headache, muscle or body aches, diarrhea, cough, sore throat, congestion or runny nose, nausea or vomiting, shortness of breath or difficulty breathing, pneumonia, fatigue, and new loss of taste or smell.

In some embodiments of the presently-disclosed subject matter, the integrin inhibitor is administered to a subject who has been infected with hCoV. In some embodiments, A treatment regimen can be designed in view of a particular situation, in accordance with recommendations from one of ordinary skill in the art. In some embodiments, the integrin inhibitor is administered multiple times over a treatment period. For example, in some embodiments, the integrin inhibitor is administered to the subject about 3 times a week to about 21 times a week. In this regard, in some embodiments, administration occurs on 3, 4, 5, 6, or 7 days of a week, and in other embodiments, administration occurs once, twice, or three times each day of a week. Furthermore, in some embodiments the treatment period could be one week, two weeks, three weeks, 4 weeks, or more. In some embodiments, the treatment period could be 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days. In some embodiments, the treatment period is ongoing until the at least one symptom has resolved. In some embodiments, the treatment period is ongoing until 24 hours after the at least one symptom has resolved. In some embodiments, the treatment period is ongoing until 1, 2, 3, 4, 5, 6, 7, 8, 9, 10, 11, 12, 13, or 14 days have passed since the at least one symptom has resolved.

As disclosed herein, the presently-disclosed invention provides for treatment that reduces viral activity in the subject. Such viral activity can include, for example, viral load, viral replication, and viral infection. Furthermore, the presently-disclosed invention provides for treatment that reduces detrimental histopathological changes, such as changes in lung. Assessment of histopathological status of tissue can be made using techniques known to a pathologist of ordinary skill in the art.

As will be appreciated by the skilled artisan upon study of the present document, the presently-disclosed subject matter includes supplemental treatment to improve infection status and/or disease state, and can be used in connection with other therapies for hCoV infections. Such therapies can be, for example, therapeutics that serve as decoys or otherwise disrupt the process by which hCoV enters a target host cell.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Ability of ATN-161 to Disrupt Binding Events Essential to Entry of SARS-CoV-2 into a Host Cell Assessed by Enzyme-Linked Immunosorbent Assay (ELISA)

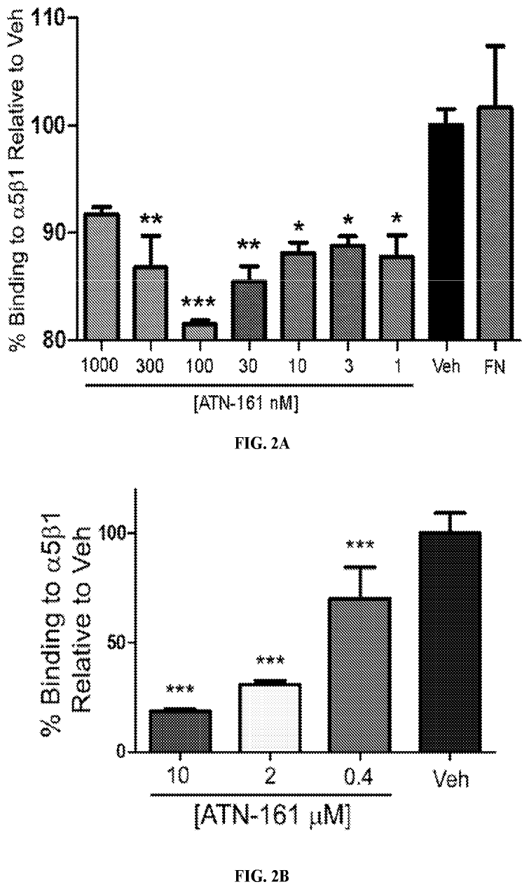

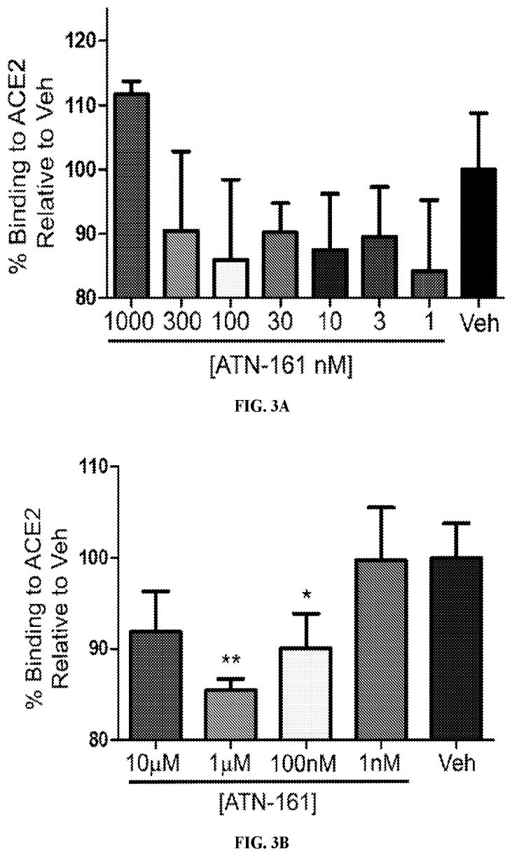

α5β1 was coated on 96-well plates at 1 μg/mL for 2 hours at room temperature and blocked overnight with 2.5% BSA. For determination of inhibition of binding between ACE2 and α5β1 integrin by ATN-161, 0.5 μg/mL of hACE2-Fc in differing concentrations of ATN-161 were added to an α5β1-coated plate, which was incubating for 1 hour at 37° C. Incubation with a horseradish peroxidase (HRP) labeled goat anti-mouse Fc secondary antibody at 1:5000 for 30 minutes at 37° C. was followed by detection by TMB substrate. Data were normalized to no-ATN vehicle control. Results are provided in FIG. 2A.

In order to assess disruption of binding of α5β1 to SARS-CoV-2 Spike protein, various concentrations of ATN-161 with 1 μg/mL spike in the presence of 1 mM MnCl₂ were added to an α5β1-coated plate. Binding was detected using an anti-spike antibody. Data were normalized to no-ATN vehicle control. Results are provided in FIG. 2B.

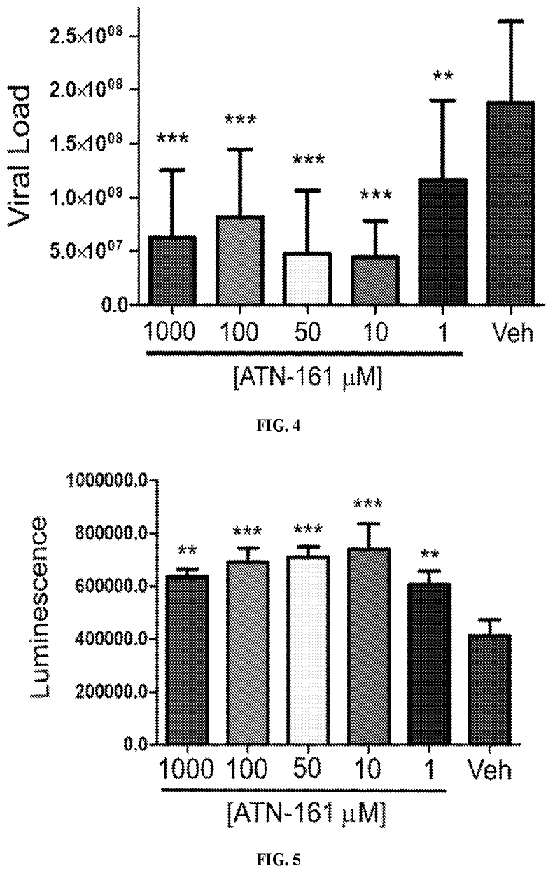

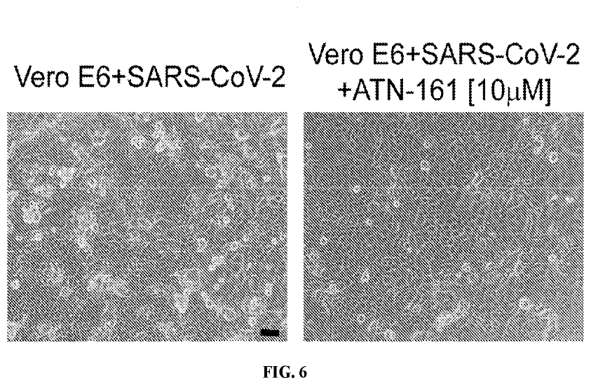

Studies were conducted to assess disruption of binding between ACE and trimeric spike protein. Plates were pre-coated with trimeric spike protein and incubated with a mixture of hACE2 and various concentrations of ATN-161 followed by detection of bound hACE2 via HRP-conjugated anti-ACE2 antibody. Data were normalized to no-ATN vehicle control. Results are provided in FIG. 3A.

Studies were conducted to assess disruption of binding between ACE and monomeric spike protein. Plates were pre-coated with monomeric spike protein and incubated with a mixture of hACE2 and various concentrations of ATN-161 followed by detection of bound hACE2 via HRP-conjugated anti-ACE2 antibody. Data were normalized to no-ATN vehicle control. Results are provided in FIG. 3B.

In this study, the binding of the viral structural protein, spike, with ACE2 and α5β1 were assessed using ELISA-based methods. To determine spike protein's ability to bind α5β1, plates were coated with α5β1 and incubated with a mixture of ATN-161 and a trimeric version of spike protein. Spike protein binds to α5β1 and ATN-161 inhibits that binding in a dose-dependent manner, with the most effective dose tested being 100 nM (FIG. 2A).

To investigate ACE2 binding with α5β1, a similar ELISA protocol was performed, with the exception of α5β1 incubation with a mixture of ATN-161 and human ACE2 protein (hACE2). Inhibition of ACE2/α5β1 binding by ATN-161 was demonstrated in a dose-dependent fashion as well (FIG. 2B). Utilizing a similar approach, ATN-161 does not reduce binding of trimeric spike protein to hACE2 but does reduce binding of monomeric spike (FIGS. 3A and 3B).

### Example 2: In vitro assessment of ATN-161 Inhibition of SARS-CoV-2 Infection

In order to determine the ability of ATN-161 to reduce the infection capability of SARS-CoV-2 in vitro, a cell-based assay was utilized. VeroE6 cells (ATCC #CRL-1586) were cultured in complete DMEM containing 10% fetal bovine serum (FBS). SARS-CoV-2 stock was obtained by infecting nearly confluent monolayers of VeroE6 cells for one hour with a minimal amount of liquid in serum free DMEM. Once adsorption was complete, complete DMEM containing 2% FBS was added to the cells and the virus was allowed to propagate at 37° C. in 5% CO₂. Upon the presence of CPE in the majority of the monolayer, the virus was harvested by clearing the supernatant at 1,000×g for 15 minutes, aliquoting and freezing at −80° C.

VeroE6 cells were plated at a density of 1.25×10⁴ cells/well in a 96-well plate and incubated overnight at 37° C. in 5% CO₂. The next day, cells were treated with dilutions of ATN-161 in complete DMEM with 2% FBS for one hour at 37° C. in 5% CO₂, followed by viral infection at an MOI of 0.1. After 48 hours, virus and cells were lysed via Trizol LS and RNA was extracted using a Zymo Direct-zol 96 RNA Kit (#R2056) according to manufacturer's instructions. Experiments were performed under Biosafety Level 3 conditions in accordance with institutional guidelines.

Viral load was quantified using a Reverse Transcriptase qPCR targeting the SARS-CoV-2 nucleocapsid gene. RNA isolated from cell cultures was plated in duplicate and analyzed in an Applied Biosystems 7300 using TaqPath supermix with the following program: i) 50° C. for 15 min., ii) 95° C. for 2 min. and iii) 45 cycles of 95° C. for 3 s and 55° C. for 30 s. The primers and probes were as follows: 2019-nCoV_N1 Forward:5′-GAC CCC AAA ATC AGC GAA AT-3′ (SEQ ID NO: 2), 2019-nCoV_N1 Reverse: 5′-TCT GGT TAC TGC CAG TTG AAT CTG-3′ (SEQ ID NO: 3), and 2019-nCoV_N1 Probe: 5′-FAM-ACC CCG CAT TAC GTT TGG ACC-BHQ1-3′ (5′-FAM-SEQ ID NO: 4-BHQ1-3′). Standard curves were generated for each run using a plasmid containing SARS-CoV-2 nucleocapsid gene (Integrated DNA Technologies, USA).

Cell imaging was conducted as follows. The day before infection, Nunc LabTek II chamber slides (Thermo, USA) were seeded with 2.5×10⁴ cells per chamber. On the day of infection, chambers were treated with dilutions of ATN-161 in complete DMEM with 2% FBS for one hour prior to infecting with SARS-CoV-2 at an MOI of 0.1. Slides were placed in a 37° C. 5% CO₂ incubator for 48 hours prior to imaging via phase contrast using an EVOS XL inverted microscope (Thermo, USA).

Ability of ATN-161 to increase cell viability was performed with CellTiterGlo (Promega, USA). Cell supernatant was removed 24 hours post infection and cells were lysed via pre-mixed CellTiterGlo reagent. Cells were incubated for 15 minutes and allowed to shake briefly before ATP was quantified via luminescence readout on the GloMax Explorer multimode plate reader (Promega).

Differences between groups was determined via the one-way ANOVA using Dunnett's multiple comparisons test. Experiments are represented as weighted mean and standard deviation of a total of three replicates. For IC₅₀ estimation, the data points directly bounding the IC₅₀ value were used and calculation was made in GraphPad Prism.

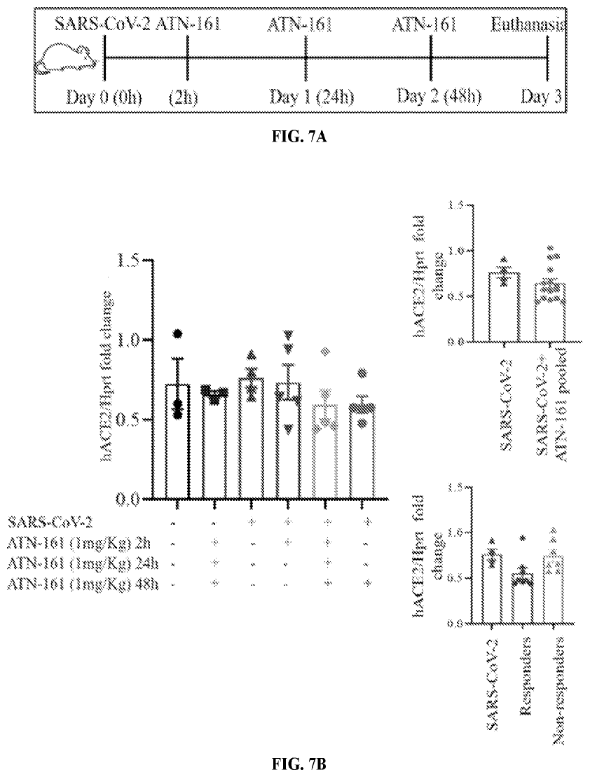

Assessing the therapeutic potential of ATN-161 was done on the VeroE6 African green monkey cell line utilizing competent SARS-CoV-2. Various concentrations of ATN-161 were incubated on cells for one hour prior to addition of virus at an MOI of 0.1. Following a 48-hour post-infection period, cells and supernatant were lysed, and RNA was extracted. PCR analysis was used to determine viral load under different conditions. ATN-161 was effective at reducing viral loads after infection (FIG. 4), with an estimated IC₅₀ of 3.16 μM. This is similar to recent values found for inhibition with potential therapeutics such as remdesivir⁸.

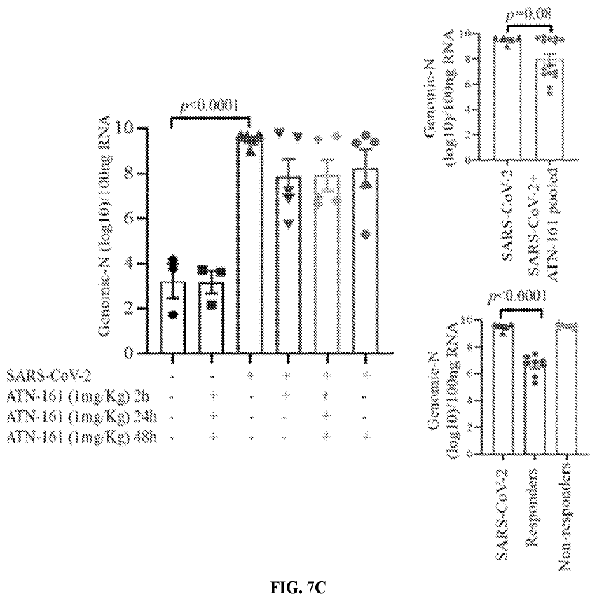

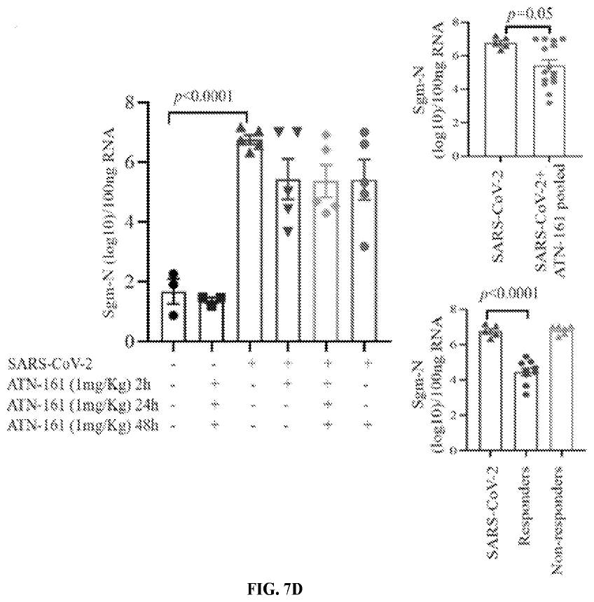

Measuring cellular viability is another metric for anti-viral therapeutic potential that was explored with ATN-161. After infecting cells at an MOI of 0.01 for 24 hours, cells were lysed with CellTiterGlo and luminescence values were taken to measure ATP production in each treatment. Consistent with the PCR data, pre-treatment with ATN-161 increased ATP production in infected cells, indicating increased viability. This was apparent at all doses of ATN-161, but most apparent in the 10 μM treatment (FIG. 5). This is seen as well when cell monolayers are visualized using phase contrast microscopy (FIG. 6), with treatment with ATN-161 at 10 μM resulting in decreased evidence of cytopathic effect (i.e. fewer apparent rounded, phase bright cells).

SARS-CoV-2 spike protein binds to α5β1 and this binding, as well as that of α5β1/hACE2, can be inhibited effectively by ATN-161, as well as infection by SARS-CoV-2 in vitro. This was further supported by cellular viability increases in ATN-161-treated infected cells, as well as decreased cytopathic effect as determined visually.

### Example 3: In Vivo Protection from SARS-CoV-2 Infection by ATN-161 in k18-hACE2 Transgenic Mice

SARS-CoV-2 Infection. Mice were inoculated with either saline or SARS-CoV-2 via intranasal administration by the ABSL3-trained staff with a dose of 2×10⁵ TCID50/mouse to induce viral infection in these animals [35, 36]. The infected mice were observed daily to record body weight and clinical signs of illness (e.g. fur ruffling, less activity). After 3 days post infection (dpi), the mice were euthanized by CO₂ asphyxiation followed by cervical dislocation and lungs were harvested for histology, immunofluorescence and qRT-PCR analysis.

Treatment Interventions. Mice received ATN-161 (1 mg/kg, Medkoo Biosciences, Morrisville, N.C., USA) via retro orbital i.v. injections. The subjects were divided into 6 groups; two control groups were non-infected: 1. saline treated control (saline, n=3) and 2. ATN-161 treated mice (ATN-161, n=3). These mice resided in the BSL1 portion of the Tulane Department of Comparative Medicine animal housing facility. Four SARS-CoV-2 infected groups received either saline or ATN-161 prepared fresh daily (3. SARS-CoV-2+saline (n=5), 4. SARS-CoV-2+ATN-161 once at 2 h (n=5), 5. SARS-CoV-2+ATN-161 daily (2, 24, and 48 h, (n=5)) and 6. SARS-CoV-2+ATN-161 once at 48 h (n=5) treatment administration following intra nasal viral inoculation with SARS-CoV-2.

Viral copy number determination. Tissues were weighed and homogenized in Trizol Lysis Reagent (Invitrogen). RNA was extracted from lung homogenates according to the instructions of the RNA extraction kit manufacturer (RNeasy Plus Mini Kit; Qiagen) post phase separation using Trizol reagent. Total RNA (Five microliters) was added in duplicate to a 0.2-ml standard 96-well optical microtiter plate (Cat. No. N8010560; Thermo Fisher). qRT-PCR reaction was set by using TaqPath 1-Step Master Mix (Cat. No. A28527; Thermo Fisher) with a primers and a FAM-labeled probe targeting the N1 amplicon of N gene (2019-nCoV RUO Kit, Cat. No. 10006713; IDT-DNA) of SARS-CoV-2 (https://www.ncbi.nlm.nih.gov/nuccore; accession number MN908947), following the manufacturer's instructions. Viral load was calculated by the linear regression function by Cq values acquired from 2019 nCoV qRT-PCR Probe Assays. The viral copy numbers from the lung samples are represented as copies/100 ng of RNA as followed using a published assay [37, 35, 38]. Subgenomic mRNA (sgmRNA) encoding the E gene was quantified using FAM-labeled primers (sgm-N-for: 5′-CGATCTCTTGTAGATCTGTTCTC-3′ (SEQ ID NO: 5), sgm-N-Probe:5′-FAMTAACCAGAATGGAGAACGCAGTGGG-TAMRA-3′ (5′-FAM-SEQ ID NO: 6-TAMRA-3′), sgm-N-reverse:5′-GGTGAACCAAGACGCAGTAT-3′ (SEQ ID NO: 7)), following the manufacturer's instructions. Subgenomic N viral copy number was calculated by standard Cq values. The viral copy numbers from the lung samples are represented as copies/100 ng of RNA followed using a published assay [38, 35].

Gene expression. The lung tissue was homogenized in Trizol Lysis Reagent (Invitrogen). RNA was extracted from lung homogenates according to the instructions of the RNA extraction kit manufacturer (RNeasy Plus Mini Kit; Qiagen) post phase separation using Trizol reagent. RNA was converted to cDNA using iScript reverse transcriptase master mix (Bio-Rad). qPCR was carried out with QuantStudio 3 Real-Time PCR Systems (Life Technologies) using TaqMan PCR Master Mix and premixed primers/probe sets (Thermo Fisher Scientific) sets specific for Itgav (Mm00434486 ml), Cxcl10 (Mm00445235_m1), Itga5 (Mm00439797_m1), Itgb3 (Mm00443980_m1), Itgb1 (Mm01253233_m1), ACE2 (Hs01085333_m1), and Hypoxanthine phosphoribosyltransferase, Hprt (Mm01545399_m1, Control gene) (Life Technologies) were used gene expression. Data were analyzed comparing control to SARS-CoV-2 infected mice and are presented as a fold change of control.

Histology. The harvested whole right lungs (three lobes) were fixed in Z-fix (Anatech Ltd, Battle Creek, Mich., USA). Paraffin sections (5 μm in thickness) were used for haematoxylin and eosin and Masson's trichrome stains to identify morphological changes in lungs. Slides were imaged with a digital slide scanner (Zeiss Axio Scan; Zeiss, White Plains, N.Y.). Representative photo micrographs at 20× magnification were acquired from the whole scanned right lung using the Aperio Image Scope software (version 12.3.2.8013, Leica, Buffalo Grove, Ill., USA).

SARS-CoV-2 immunofluorescence. Fixed tissue samples were processed via indirect immunofluorescence assays (IFA) for the detection of the SARS-CoV-2 antigen. The slides were deparaffinized in xylenes and rehydrated through an ethanol series, followed by heat-induced antigen retrieval with high pH antigen unmasking EDTA solution. The slides were washed with PBS with 0.1% Triton X-100 and blocked with 10% normal goat serum at room temperature for 1 h. Primary antibody (Polyclonal Anti-SARS Coronavirus (antiserum, Guinea Pig) 1:1000, NR-10361) incubation was achieved at room temperature for 1 h. Slides were washed and primary antibody detected following 40 min incubation in an appropriate secondary antibody tagged with Alexa Fluor fluorochromes (1:1000) in normal goat serum. After washing in PBS, mounting media with DAPI was used to label the nuclei. Slides were imaged with a digital slide scanner (Zeiss Axio Scan.Z1; Zeiss, White Plains, N.Y.). Fluorescent images were acquired using HALO (Indica Labs, v2.3.2089.70). Threshold and multiplex analyses were performed with HALO for quantitation.

Statistics. Statistical tests were performed with GraphPad Prism, 8.4.3 version (GraphPad Software, San Diego, Calif.). Data are presented as mean±SEM. Significant differences were designated using omnibus one-way ANOVA and, when significant, followed-up with two-group planned comparisons selected a priori to probe specific hypothesis-driven questions (saline vs SARS-CoV-2+vehicle; SARS-CoV-2+vehicle vs each of the various SARS-CoV-2+ATN-161 treated groups); the Holm-Sidak adjustment was applied to control for multiple comparisons. Non-parametric tests were employed in the event of violations of underlying ANOVA assumptions. Statistical significance was taken at the p<0.05 level.

ATN-161 impacts the Genomic-N and Sub genomic N (Sgm-N) viral load in SARS-CoV-2 infected K18-hACE2 mice lung tissue.

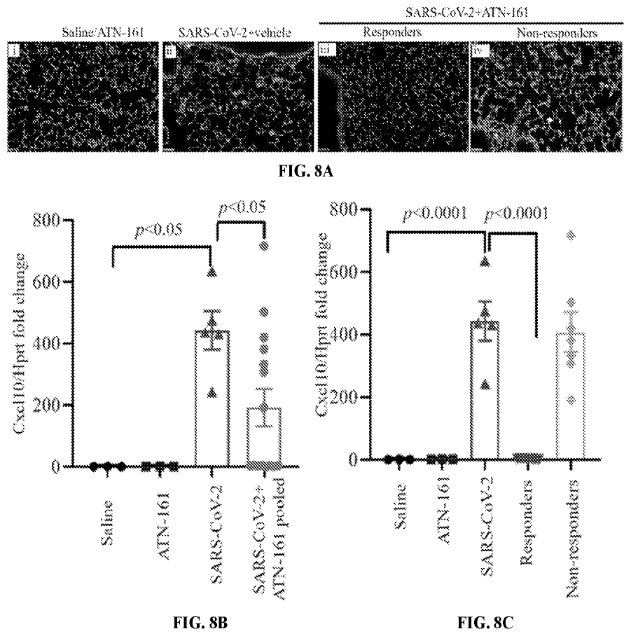

Lung tissue expression of hACE2 and viral loads after 3 dpi with SARS-CoV-2+Saline or ATN-161 administered either once at 2 h or 48 h post-infection or daily, post-infection, were measured, analyzed and compared to Vehicle-treated/uninfected (no SARS-CoV-2 exposure) mice either given Saline or ATN-161 (daily) for 3 dpi. (FIG. 7A).

With reference to FIG. 7B-7D (large bar graphs), 10-week old male K18-hACE2 transgenic mice administered saline (first bar) or ATN-161 (1 mg/kg, second bar) intravenously (via retro-orbital). Mice were inoculated via the intranasal route with Severe acute respiratory syndrome coronavirus 2 (SARS-CoV-2) (2×10⁵ TCID50)+Saline (i.v. by retroorbital rout) (third bar). ATN-161 (1 mg/Kg) treatment was administered at 3 different time periods, SARS-CoV-2+ATN-161-2 h post infection (fourth bar), SARS-CoV-2+ATN-161-daily (2 h, 24 h and 48 h) administration (fifth bar), and SARS-CoV-2+ATN-161-48 h administration (sixth bar) post SARS-CoV-2 intranasal inoculation. With reference to the top inserts of FIG. 7B-7D, the second bar represents SARS-CoV-2+ATN-161 all treatment groups pooled data. With reference to the bottom inserts of FIG. 7B-7D, the second bar represents responding mice and third bar represents non responding with SARS-CoV-2+ATN-161 (either 2, daily or 48 h) treatment. 3 days post infection (3 dpi) the mice were euthanized, and RNA isolated from the left lungs by Trizol method for qRT-PCR. FIG. 7B shows the resulting expression in lungs of K18-hACE2 mice, FIG. 7C shows the resulting viral genomic-N(Total-N) mRNA, and FIG. 7D shows the resulting sub-genomic-N mRNA (sgm-N).

For hACE2, the omnibus one-way ANOVA was not significant (FIG. 7B). For Genomic-N(FIG. 7C), the omnibus one-way ANOVA was significant [F(5,20)=12.84, p<0.0001]. This analysis was followed up with two group planned comparisons using the Holm-Sidak correction. A significant increase was found in Genomic-N among SARS-CoV-2+Saline mice compared to non-infected, Saline-treated mice [46)=5.98, p<0.0001]. Though significant group differences were not detected between any other two-group comparison, visual inspection of the graph revealed that there was 1) heterogeneity among the ATN-161 treated groups, suggesting a dichotomy in this population with regards to response to the ATN-161 treatment with regard to the viral load and 2) a general trend towards reduced viral load among all ATN-161 treated groups, regardless of timepoint or number of injections. This indicated to us a potential for the analyses to be underpowered. Therefore, to increase power and reduce variability, all ATN-161 treated animals were pooled into a single group and re-analyzed the data by comparing lung viral load in this group to that of the SARS-CoV-2+Saline-treated mice. A trend towards significance [418)=2.03, p<0.06] was found such that ATN-161-treated mice had lower Genomic N viral load in lungs than SARS-CoV-2-infected mice. The data were analyzed using the Mann-Whitney U nonparametric test to account for a significant (p<0.01) violation of the homogeneity of variance assumption (though ANOVA is robust to this type of violation), the effect remained marginal (p=0.08). ATN-161-treated animals were dichotomized into ‘responder’ and ‘non-responder’ groups such that mice were considered non-responders if they displayed Genomic N values >2×10⁹ (one power of 10 lower than the lowest value observed in the SARS-CoV-2 group), Sgm-N values >1×10⁵ (one power of 10 lower than the lowest value observed in the SARS-CoV-2 group), and viral immunohistology staining counts >0.7 (one power of 10 lower than the lowest value observed in the SARS-CoV-2 group). Ns of responders for each ATN-161 treated group were 3, 3, and 2 for ATN-161-2 hr, ATN-161-daily, and ATN-161-48 hr groups respectively. When the data was re-analyzed by comparing lung viral load in these groups to that of the SARS-CoV-2+Saline-treated mice, a significant omnibus one-way ANOVA [F(2,17)=35.32, p<0.0001] was found, such that ATN-161 responders had significantly lower genomic lung viral loads than SARS-CoV-2-infected animals [t(17)=7.04, p<0.0001].

For Sgm-N(FIG. 7D), the omnibus one-way ANOVA was significant [F(5,20)=13.81, p<0.0001] such that there was a significant increase in Sgm-N among SARS-CoV-2+Saline mice compared to non-infected, Saline-treated mice [46)=6.13, p<0.0001]. Analysis of Sgm-N values among pooled ATN-161-treated mice again revealed that ATN-161 reduced Sgm-N viral load [418)=2.23, p<0.05] that persisted (p=0.05) when the Mann-Whitney U non-parametric analysis was applied given the significant (p<0.05) homogeneity of variance in this group. When the data was re-analyzed by comparing lung viral load in the responder/non-responder groups to that of the SARS-CoV-2+Saline-treated mice, a significant omnibus one-way ANOVA [F(2,17)=39.91, p<0.0001] was found such that ATN-161 responders had significantly lower Sgm-N lung viral loads than SARS-CoV infected animals [t(17)=7.40, p<0.0001].

ATN-161 effects on the expression of virus in SARS-CoV-2 infected K18-hACE2 mice lung tissue.

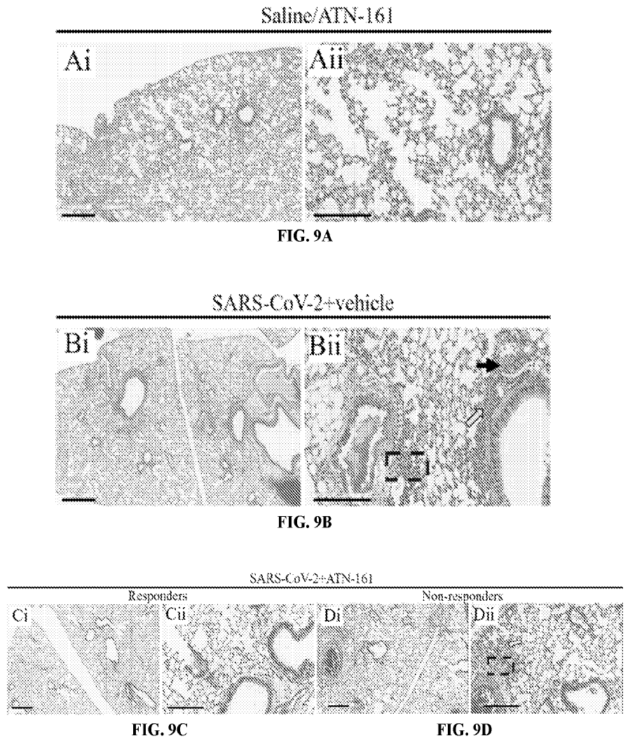

Given the observations, the analyses was continued to compare non-infected Saline or ATN-161-treated mice with SARS-CoV-2+Saline and either pooled ATN-161-treated infected mice or ATN-161 responders/non-responders. Immunohistochemistry staining for SARS-CoV-2 positive cells was conducted on all mice. Representative SARS-CoV-2 viral staining images from: (i) saline/ATN-161, (ii) SARS-CoV-2+vehicle (iii) responders, and (iv) non-responders with SARS-CoV-2+ATN-161 administration either 2, daily or 48 h administration, post SARS-CoV-2 intranasal inoculation are depicted in FIG. 8A. All K-18 hACE2 mice infected with SARS-CoV-2+Saline or non-responders from SARS-CoV-2+ATN-161 administration group had multifocal regions of SARS-CoV-2— positive cells (ii, iv), whereas treatment with ATN-161, 2 h and daily administered groups show 3 mice are completely negative for SARS-CoV-2 protein and 2 mice are negative (responders) in 48 h ATN-161 group out of 5 mice challenged for SARS-CoV-2.

One-way ANOVA of C-X-C motif chemokine ligand 10 (Cxcl10) mRNA expression in lung comparing saline or ATN-161 treated non-infected, SARS-CoV-2+Saline, and SARS-CoV-2+ATN-161 pooled mice (FIG. 8B) revealed a significant effect [F(3,23)=4.56, p<0.05] such that SARS-CoV-2+Saline mice had significantly higher lung Cxcl10 mRNA expression compared to Saline-treated uninfected mice [46)=3.07, p<0.05] and pooled ATN-161-treated mice [418)=2.46, p<0.05]. One-way ANOVA of Cxcl10 mRNA expression in lung comparing saline or ATN-161 treated non-infected, SARS-CoV-2+Saline, and SARS-CoV-2+ATN-161 responders and non-responders (FIG. 8C) revealed a significant effect [F(4,21)=24.38, p<0.0001], that persisted when the Kruskal-Wallis non-parametric analysis (p<0.005) was applied given the significant violation of homogeneity of variance (p<0.05) in this ANOVA analysis. Two-group planned follow-up comparisons revealed that SARS-CoV-2+Saline mice had significantly higher lung Cxcl10 mRNA expression compared to Saline-treated uninfected mice [46)=5.59, p<0.0001] and ATN-161-treated responders [t(11)=7.12, p<0.0001].

Histopathological Analysis of SARS-CoV-2 infected K18-hACE2 mice lung tissue by Hematoxylin and eosin stain.

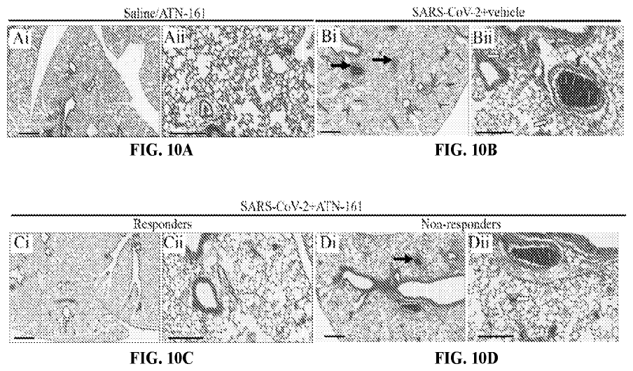

Representative images of hematoxylin and eosin staining of lung sections from saline/ATN-161 (FIG. 9A); SARS-CoV-2+vehicle (FIG. 9B); Responders (FIG. 9C) and non-responders (FIG. 9D) with SARS-CoV-2+ATN-161 (either 2 h, daily, or 48 h) administration post-SARS-CoV-2 intranasal inoculation are shown in FIG. 9A-9D. Histopathological observations indicated that multifocal lesion, moderate interstitial pneumonia (FIG. 9B(ii), 9D(ii), black frames), infiltration of lymphocytes (FIG. 9B(ii), 9D(ii), white arrow), fibrin exudation (FIG. 9B(ii), black arrow) were observed in SARS-CoV-2+vehicle and SARS-CoV-2+ATN-161 non responders mice whereas these observations are less or completely absent in SARS-CoV-2+ATN-161 responders mice. Hematoxylin and eosin staining of lung sections from saline/ATN-161 (FIG. 9A) revealed no histopathological changes in any of the mice.

Histopathological Analysis of SARS-CoV-2 Infected K18-hACE2 Mice Lung Tissue Masson's Trichrome Stain.

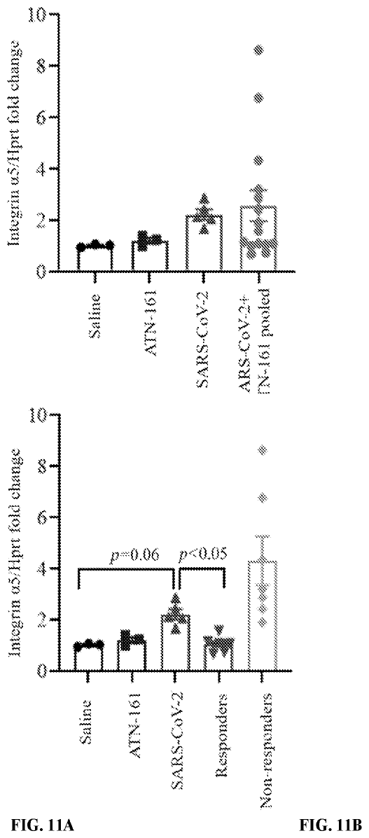

Representative images of Masson's Trichrome-stained sections (FIG. 10A-10D) of lung sections from saline/ATN-161 (FIG. 10A); SARS-CoV-2+vehicle (FIG. 10B); Responders (FIG. 10C) and Non-responders (FIG. 10D) with SARS-CoV-2+ATN-161 (either 2 h, daily, or 48 h) administration post SARS-CoV-2 intranasal inoculation. Histopathological analysis revealed multiple intra-arteriolar microthrombi (black arrows), intra-alveolar microthrombi (white arrows), large interstitial hemorrhagic area (white arrow) in SARS-CoV-2+vehicle and SARS-CoV-2+ATN-161 non responders mice whereas these observations are less or completely absent in SARS-CoV-2+ATN-161 responders mice. Masson's Trichrome staining of lung sections from saline/ATN-161 (FIG. 10A) were no histopathological changes in any of the mice.

Induced Expression of Integrin α5, and Integrin αv in the Lungs of SARS-CoV-2 Infected K18-hACE2 Mice and is Inhibited by ATN-161 Treatment.

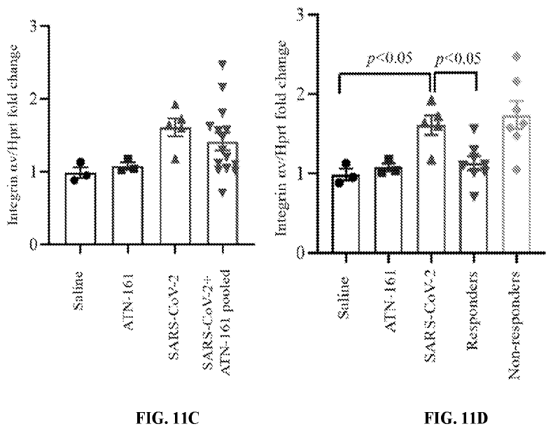

One-way ANOVA of integrin-α5 expression in lung comparing saline or ATN-161 treated non-infected, SARS-CoV-2+Saline, and SARS-CoV-2+ATN-161 pooled mice was not significant (FIG. 11A). One-way ANOVA of integrin-α5 expression in lung comparing saline or ATN-161 treated non-infected, SARS-CoV-2+Saline, and SARS-CoV-2+ATN-161 responders and non-responders (FIG. 11B) revealed a significant effect [F(4,21)=6.59, p<0.05], that persisted when the Kruskal-Wallis non-parametric analysis (p<0.05) was applied given the trend towards a violation of homogeneity of variance in this ANOVA analysis (p=0.06). Because there was very high variability in the SARS-CoV-2+ATN-161 non-responders, and there was concern about the possibility of inflated Type 1 error risk, two-group follow-up comparisons were constructed using the non-parametric approach (Dunn-corrected). It was found that SARS-CoV-2+Saline mice tended to have higher lung integrin-α5 expression compared to uninfected Saline-treated mice [z=2.32, p=0.06].

One-way ANOVA of integrin-αv expression in lung comparing saline or ATN-161 treated non-infected, SARS-CoV-2+Saline, and SARS-CoV-2+ATN-161 pooled mice was not significant (FIG. 11C). One-way ANOVA of integrin-αv expression in lung comparing saline or ATN-161 treated non-infected, SARS-CoV-2+Saline, and SARS-CoV-2+ATN-161 responders and non-responders (FIG. 11D) revealed a significant effect [F(4,21)=6.04, p<0.005], such that SARS-CoV-2+Saline mice had significantly higher lung integrin-αv expression compared to Saline-treated uninfected mice [46)=2.70, p<0.05] and ATN-161-treated responders [(11)=2.67, p<0.05].

The coronavirus disease-2019 (COVID-19) caused by SARS-CoV-2 continues to ravage the world. As of Mar. 24, 2021, there were 3,089,162 deaths with a total of 145,759,060 confirmed (COVID-19) cases worldwide [39, 40]. While the development of vaccines and antibody treatments against COVID-19 is promising and globally accepted in the current pandemic [41], the emergence of SARS-CoV-2 viral variants may present major challenges to these therapeutic approaches. Like most viruses, SARS-CoV-2 mutates and continually presents variants. Spike protein mutations are the most common mutation seen in SARS-CoV-2 variants, which facilitate viral entry into the cell and mediate viral propagation by binding to ACE2 receptor. Interestingly, an RDG integrin-binding motif is a novel feature of SARS-CoV-2 spike protein, which is not seen in other coronaviruses [42, 11]. While this feature may have enhanced viral infectivity of SARS-CoV-2, it remains unknown how variant strains of SARS-CoV-2 may affect binding to integrins, with the resultant feature of affecting viral entry and propagation. However, none of the currently characterized SARS-CoV-2 variants have been found to directly mutate the RGD motif. This suggests the possibility that the RGD motif is of evolutionary advantage to the virus by supporting its ability to infect hosts.

Integrins are cell surface receptors that may bind to the SARS-CoV-2 spike protein interaction [43, 44, 11, 34]. With reference to Example 1., it was demonstrated by ELISA that the SARS-CoV-2 spike protein and ACE2 could bind to α5β1, and that ATN-161 could disrupt both of these interactions [27]. With reference to Example 2, ATN-161 was also found to significantly reduce SARS-CoV-2 infection (viral load, cell viability and cytopathy) in Vero (E6) cells in vitro [27]. In this Example, the understanding of the role of integrins was extended and the therapeutic role of ATN-161 against SARS-CoV-2 infection was explored in vivo in the k18-hACE2 mice model.

K18-hACE2 mice provide a platform for the rigorous screening of candidate drugs before their evaluation in other animal models [45]. This mouse model is widely used to evaluate the pathogenicity of viruses such as SARS-CoV-2 that require or prefer the human form of ACE2 (versus mouse ACE2) to readily infect mice and can be used to study potential therapies [46, 36]. Previous studies provided the evidence that SARS-CoV-2 infection could cause typical interstitial pneumonia and develop respiratory disease in hACE2-expressing mice resembling what is commonly seen in COVID-19 patients [47, 46, 48, 49].

Genomic-N(Nucleocapsid (N) protein) and Sgm-N were measured in lungs to assess SARS-CoV-2 viral infectivity and replication. The results showed that SARS-CoV-2— infected hACE2 mice had significantly higher SARS-CoV-2 genomic-N. Similarly, viral sgmRNA copies were detected predominantly in the lung as compared to uninfected saline treated mice. SgmRNA levels of the virus is an adequate surrogate assay for detection of replicating virus (replicating virus separated from the total genome, and form dimers as the virus is replicating its machinery which can continue to produce protein) [35]. Thus, it was determined that the mice were successfully infected with SARS-CoV-2 [36].

It was found that ATN-161-treated mice had lower Genomic-N viral load in lungs than saline treated SARS-CoV-2-infected mice. Visual inspection of Genomic-N and sgm-N graph revealed that there was 1) heterogeneity among the ATN-161 treated groups, suggesting a dichotomy in this population with regards to ATN-161 treatment response and viral load, and 2) a general trend towards reduced viral load among all ATN-161 treated groups, regardless of timepoint or number of injections. A limitation of this study is that it employed a limited number of K18 hACE2 mice, a function of the logistical difficulty of doing live virus BSL-3 studies. ATN-161-treated animals were also dichotomized into ‘responder’ and ‘non-responder’ groups such that mice were considered non-responders if they displayed Genomic N values >2×10⁹, Sgm-N values >1×10⁵ and viral immunohistology staining counts >0.7 values observed in the SARS-CoV-2 group in the SARS-CoV-2+saline group. ATN-161 responders had significantly lower genomic lung viral loads than SARS-CoV-2-infected animals.

Accordingly, this bimodal distribution of responders may be due to the use of heterozygous (HT) K18-hACE2 mice, as the K18-hACE2 homozygous mouse model completely replaces mACE2 expression with hACE2 under the mAce2 promoter [50]. Thus, it might be possible that the use of HT K18-hACE2 mice in this study produced variable expression patterns of mACE2 vs hACE2, reducing the efficacy of ATN-161 in mice that expressed higher levels of mACE2 relative to hACE2. ATN-161 interference with mACE2-α5β1 interactions would presumably have minimal to no effect on SARS-CoV-2 infection as compared to impacting hACE2-α5β1 interactions. ATN-161 reduced Sgm-N viral load among pooled ATN-161-treated mice. When the data was re-analyzed by comparing lung viral load in the responder/non-responder groups to that of the SARS-CoV-2+Saline-treated mice, it was found that ATN-161 responders had significantly lower Sgm-N lung viral loads than SARS-CoV-2-infected animals. This suggests a lack of statistical power due to low Ns among individual ATN-treated groups; follow-up studies with additional subjects will be conducted to replicate these findings.

All K-18 hACE2 mice infected with SARS-CoV-2 or non-responders from SARS-CoV-2+ATN-161 administered group had multifocal regions of SARS-CoV-2— positive cells. The results are further supported by recent findings of lung histological changes in SARS-CoV-2 infected hACE2 mice [36, 50]. Furthermore, it was observed that viral immunohistology staining counts were negative in responders for each ATN-161 treated group with n's of 3, 3, and 2 for ATN-161-2 h, ATN-161-daily, and ATN-161-48 h groups, respectively. The infected K18-hACE2 mice did not lose body weight after only 3 dpi as expected, but high levels of viral copies were observed and infectious virus in the lungs given as demonstrated by other studies in K18-hACE2 mice [36, 50, 51].

In regards to an inflammatory response to SARS-CoV-2 infection, it was observed that Cxcl10 mRNA expression was significantly upregulated in the lungs of SARS-CoV-2 infected mice as has previously been reported [51]. In patients having rapid early viral replication, this is followed by inflammatory responses that contribute to pathology [52]. Post-mortem analysis of human COVID-19 patients showed immune cell accumulation in the lungs [53]. In the study, ATN-161 treatment, either pooled or ATN-161 responders, significantly lowered SARS-CoV-2-induced Cxcl10 levels, a particularly robust result suggesting that ATN-161 has anti-inflammatory properties in the context of SARS-CoV-2 infection. This result is in agreement with previous studies in experimental ischemic stroke where post-stroke treatment with ATN-161 reduced neuro-inflammation [31].

Morphological changes observed at 3 dpi in infected lungs of K18-hACE2 mice included multifocal lesions, moderate interstitial pneumonia, infiltration of lymphocytes, and fibrin exudation. These were seen in SARS-CoV-2+vehicle and SARS-CoV-2+ATN-161 non responders mice but were less or completely absent in SARS-CoV-2+ATN-161 responder mice as well as absent in uninfected saline/ATN-161 treated mice. These findings are consistent with previous reports from post-mortem examination of patients with COVID-19 and SARS-CoV-2-infected K18-hACE2 mice in other reports [51, 50, 54-56].

Pulmonary fibrosis, a particularly negative consequence of COVID-19-induced acute respiratory distress syndrome [57], was not prominentin infiltrated areas at 3 dpi in infected lungs of K18-hACE2 mice. However, moderate multiple intra-arteriolar microthrombi, intra-alveolar microthrombi, and large interstitial hemorrhagic areas were observed in SARS-CoV-2+vehicle and SARS-CoV-2+ATN-161 non responder mice whereas these observations were less or completely absent in SARS-CoV-2+ATN-161 responder mice. Severely infected COVID-19 patient autopsy samples showed that microthrombi or immunothrombi is associated with a hyperinflammatory response [57, 58]. The findings are further supported by recent results in the human immune system humanized mouse model on histological changes in the lungs with SARS-CoV-2 infection [59, 36, 50].

The results suggest a critical role of integrins as an additional receptor to SARS-CoV-2 spike protein cell entry [60, 61]. Induced expression of integrin α5 and integrin αv was observed in the lungs of SARS-CoV-2 infected K18-hACE2 mice whereas lung expression of hACE2 levels did not vary in SARS-CoV-2+Saline or ATN-161 treated mice, suggesting that SARS-CoV-2 infection and/or pathogenesis involves these, and perhaps other integrins, that activate downstream signaling to induce lung pathology [34, 62-64]. Indeed, a recent study showed that increased integrin α5β1 and αvβ3 levels in cardiac myocytes, obtained from heart failure patients, correlates with ACE2 expression [65]. This suggests that the concomitant elevation of these integrins and the upregulation of ACE2 in an organ may render it more susceptible to SARS-CoV-2 infection. Hence, as observed in the present study, decreasing the expressions of α5β1 and αVβ3 in the presence of ACE2 may dampen the effect of SARS-CoV-2 infection on lung tissue morphology. Other studies have clearly linked integrin α5β1, and its inhibition, with other viral infections [63, 66]. ATN-161 treatment inhibited porcine hemagglutinating encephalomyelitis virus (PHEV) infection and its increase in the expression of integrin α5β1 in vivo in a mouse model [66]. Similarly, it was observed that ATN-161 treatment inhibits SARS-CoV-2-induced integrin α5, and integrin αv in the K18-hACE2 mice lungs among ATN-responders. The studies were performed at the dose of ATN-161, 1 mg/kg, that demonstrated efficacy in in vitro and in vivo preclinical studies in blocking angiogenesis, solid tumor growth, and ischemic stroke injury [67, 31, 32]. This dose was also similar to the 0.8 mg/kg dose used in the PHEV anti-viral in vivo studies [66].

ATN-161 present several potential advantages as a novel COVID-19 therapy. Unlike other RGD-based peptides, it preferentially binds to and inhibits activated forms of α5β1 in areas of inflammation, hypoxia and angiogenesis [31]. ATN-161 also binds to integrin αvβ3 [31], an additional integrin that has been implicated in SARS-CoV-2 pathogenesis [34, 33], and is safe, well-tolerated in human clinical trials (cancer) with no dose limiting toxicity [29, 68], and can be administered i.v., i.p., and intranasally [32, 69]; The latter supports a more readily accessible means of COVID-19 treatment as well as affording a prophylactic approach.

These studies demonstrate that integrin blockade can reduce SARS-CoV-2 infection in vivo. Indeed, these studies demonstrate that post-infection treatment with ATN-161 reduces lung viral load, replication, improves lung histology, and reduces lung α5 and αv integrin expression in a majority of SARS-CoV-2 infected k-18 mice. These studies also show that inhibition of integrin α5β1 (and αvβ3) in vivo can reduce both SARS-CoV-2 viral load and pathological complications in lung tissue of SARS-CoV-2 in k18-hACE2 mice. Thus, ATN-161 is identified herein for therapeutic and prophylactic treatment after SARS-CoV-2 exposure.

To date, several small-molecule drug and peptide treatments have been developed for intranasal delivery which reach systemic circulation rapidly, many of which have been formulated to treat diseases which have the ability to alter cognition, such as depression [70]. As the olfactory and trigeminal nerves offer a safe and effective delivery pathway to deliver therapeutic agents to the brain, an intranasal-based therapy to both prevent and treat neurological complications of post-acute COVID-19 syndrome are contemplated to include a formulation of ATN-161 [71, 72].

### Example 4: In Vivo Protection from SARS-CoV-2 Infection by ATN-161 in Ad5-hACE Mice

13-week-old male wildtype C57BL/6J mice were obtained from the Jackson Laboratory. Mice were housed in the animal facility at Tulane University School of Medicine. The Institutional Animal Care and Use Committee of Tulane University reviewed and approved all procedures for sample handling, inactivation, and removal from a BSL3 containment (permit number P0443).

Mice were oropharyngeally transduced with 1.5×10⁹ plaque-forming units (PFU)/mouse of Ad5-hACE2 (Vector Biosystems, Inc.) under Animal Biosafety Level 2 (ABSL2) conditions to induce ACE2 expression in lungs. Then four days later mice were inoculated with either saline or SARS-CoV-2 via intranasal administration by the ABSL3-trained staff with a dose of 1×10⁵ TCID50/mouse to induce viral infection. The infected mice were observed daily to record body weight and clinical signs of illness.

After 3 days post infection (dpi), the mice were euthanized by CO₂ asphyxiation and lungs were harvested for histology, immunofluorescence, and qRT-PCR analysis. hACE2 expression at the transcriptional level in the lungs was determined by qRT-PCR using specific primer pairs for hACE2 (Thermo Fisher).

Data are presented as mean±SD. Significant differences were designated using omnibus one-way ANOVA and, when significant, followed-up with two-group planned comparisons selected a priori to probe specific hypothesis-driven questions (saline vs ad5-hACE2+SARS-CoV-2 and ad5-hACE2+SARS-CoV-2 vs ad5-hACE2+SARS-CoV-2+ATN-161 2 h treated group); the Holm-Sidak adjustment was applied to control for multiple comparisons. Statistical significance was taken at the p<0.05 level.

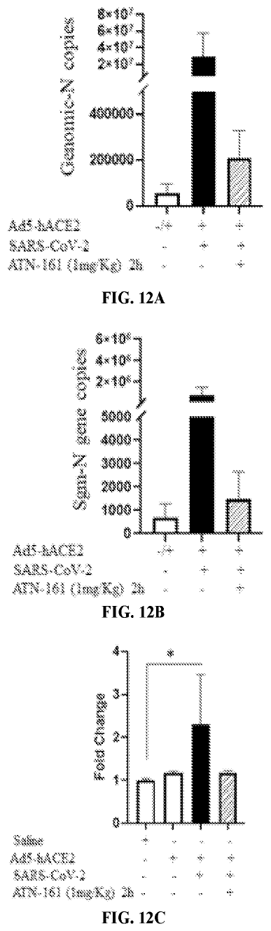

Transcriptomic analysis of lung of SARS-CoV-2 infected Ad5-hACE mice viral loads after 3 dpi with Ad5-hACE2+saline, SARS-CoV-2 infected Ad5-hACE+saline or ATN-161 administered once at 2 h pre-infection, were measured, analyzed and compared to saline-treated/uninfected (no SARS-CoV-2 exposure) mice. There was an increase in Genomic-N(FIG. 12A) and Sub genomic N (Sgm-N) (FIG. 12B) among SARS-CoV-2 infected Ad5-hACE mice compared to non-infected saline-treated Ad5-hACE mice. Whereas visual inspection of the graph indicates that ATN-161 administered 2 hours prior to SARS-CoV-2 exposure was associated with lower Genomic-N and Sub genomic N viral load in lungs. With reference to FIG. 12C, integrin alpha (a) 5 mRNA expression in lungs was analyzed by RT-PCR. Data are normalized to HPRT (house-keeping gene) within a group; fold change relative to saline-treated controls was calculated. Two-group comparisons revealed differences between SARS-CoV2 infected mice compared to mice administered Saline- or ATN-161 2 hrs prior to exposure. Experimental groups are divided into 4 groups. saline n=2; ad5-hACE2 n=2; ad5-hACE2+SARS-CoV-2 n=3; ad5-hACE2+SARS-CoV-2+ATN-161 2 h n=3. Data are presented as mean±SD. *p<0.05 represent saline vs ad5-hACE2+SARS-CoV-2 and ad5-hACE2+SARS-CoV-2 vs ad5-hACE2+SARS-CoV-2+ATN-161 2 h treatment groups.

The results revealed that SARS-CoV2 infected Ad5-hACE mice showed significantly higher α5 mRNA expression compared to mice administered saline- or ATN-161 2 hours prior to exposure.

### Example 5: Spike Proteins of SARS-CoV-2 Variants Blocked by ATN-161

The reagents used in this study were obtained through BEI Resources, NIAID, NIH, including Spike Glycoprotein Receptor Binding Domain (RBD) from SARS-Related Coronavirus 2, South Africa Variant (NR-54005) and United Kingdom Variants (NR-54004) with C-Terminal Histidine Tag, Recombinant from HEK293 Cells [73,74]. ATN-161 was obtained through Medkoo Biosciences, Morrisville, N.C.

Assays were performed to determine the ability of ATN-161 to disrupt binding to Recombinant spike (S) glycoprotein receptor binding domain (RBD) from SARS-CoV-2, South Africa variant and United Kingdom Variants proteins. Plates (96-well) were coated with 100 μl of recombinant protein in carbonated coating buffer and plates were incubated at 4° C. overnight [75].

For determination of inhibition of binding of α5β1 to the SARS-CoV-2 variant protein by ATN-161, the coated plates were blocked with 2.5% bovine serum albumin for 2 hours at room temperature. Integrin α5β1 protein (1 mg/ml) and differing concentrations of ATN-161 were added in the presence of 1 mM manganese chloride, and the plates were incubated for 1 h at 37° C. The plates were washed and incubated with anti-integrin alpha 5 antibody (1 μg/mL) for 30 min at 37° C. followed by incubation with a horse radish peroxidase labeled donkey anti-rabbit IgG secondary antibody at 1:2500 for 30 min at 37° C. Detection was conducted using 3,3′,5,5′-tetramethylbenzidine substrate as a color developer and 2 N H₂SO₄ (Sigma-Aldrich, Germany) was used to stop the reaction. Reading was conducted at 450 nm using an ELISA plate reader (Molecular Devices, USA).

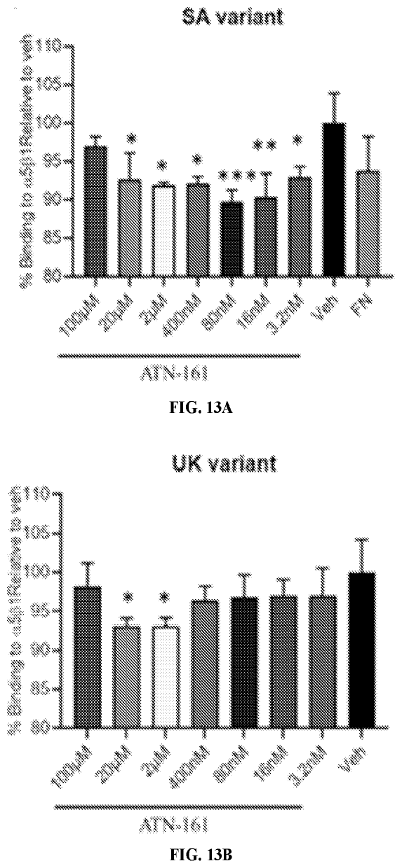

Binding of α5β1 with SARS-CoV-2 variants, including South Africa (SA) variant and United Kingdom (UK) variant, was studied using ELISA-based methods. To determine the SARS-CoV-2 variants' ability to bind α5β1, plates were coated with SARS-CoV-2 Spike RBD (SA and UK variant protein) and incubated with a mixture of α5β1 and ATN-161. The SA variant was bound to α5β1 with an affinity that was roughly equivalent to α5β1's native ligand, fibronectin, and ATN-161 inhibited binding in a U-shaped, dose-dependent manner, with maximum effect at 80 nM (FIG. 13A). Similarly, the UK variant was bound to α5β1, and ATN-161 inhibited binding with maximum effect at 2 and 20 μM (FIG. 13B).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Zhou F, Yu T, Du R, Fan G, Liu Y, Liu Z et al. Clinical course and risk factors for mortality of adult inpatients with COVID-19 in Wuhan, China: a retrospective cohort study. Lancet. 2020; 395(10229):1054-62. doi:10.1016/50140-6736(20)30566-3.
2. Carvacho I, Piesche M. RGD-binding integrins and TGF-beta in SARS-CoV-2 infections—novel targets to treat COVID-19 patients? Clin Transl Immunology. 2021; 10(3):e1240. doi:10.1002/cti2.1240.
3. Control CfD, Prevention. Interim clinical guidance for management of patients with confirmed coronavirus disease (COVID-19). 2020.
4. Perra N. Non-pharmaceutical interventions during the COVID-19 pandemic: A review. Phys Rep. 2021. doi:10.1016/j.physrep.2021.02.001.
5. Liu Y, Morgenstern C, Kelly J, Lowe R, Group CC-W, Jit M. The impact of non-pharmaceutical interventions on SARS-CoV-2 transmission across 130 countries and territories. BMC Med. 2021; 19(1):40. doi:10.1186/s12916-020-01872-8.
6. Spinelli M A, Glidden D V, Gennatas E D, Bielecki M, Beyrer C, Rutherford G et al. Importance of non-pharmaceutical interventions in lowering the viral inoculum to reduce susceptibility to infection by SARS-CoV-2 and potentially disease severity. Lancet Infect Dis. 2021. doi:10.1016/S1473-3099(20)30982-8.
7. Eubank S, Eckstrand I, Lewis B, Venkatramanan S, Marathe M, Barrett C L. Commentary on Ferguson, et al., “Impact of Non-pharmaceutical Interventions (NPIs) to Reduce COVID-19 Mortality and Healthcare Demand”. Bull Math Biol. 2020; 82(4):52. doi:10.1007/s11538-020-00726-x.
8. Xia S, Liu M, Wang C, Xu W, Lan Q, Feng S et al. Inhibition of SARS-CoV-2 (previously 2019-nCoV) infection by a highly potent pan-coronavirus fusion inhibitor targeting its spike protein that harbors a high capacity to mediate membrane fusion. Cell Res. 2020; 30(4):343-55. doi:10.1038/s41422-020-0305-x.
9. Lan J, Ge J, Yu J, Shan S, Zhou H, Fan S et al. Structure of the SARS-CoV-2 spike receptor-binding domain bound to the ACE2 receptor. Nature. 2020; 581(7807):215-20. doi:10.1038/s41586-020-2180-5.
10. Hoffmann M, Kleine-Weber H, Schroeder S, Kruger N, Herrler T, Erichsen S et al. SARS-CoV-2 Cell Entry Depends on ACE2 and TMPRSS2 and Is Blocked by a Clinically Proven Protease Inhibitor. Cell. 2020; 181(2):271-80 e8. doi:10.1016/j.ce11.2020.02.052.
11. Sigrist C J, Bridge A, Le Mercier P. A potential role for integrins in host cell entry by SARS-CoV-2. Antiviral Res. 2020; 177:104759. doi:10.1016/j.antiviral.2020.104759.
12. Yan S, Sun H, Bu X, Wan G. New Strategy for COVID-19: An Evolutionary Role for RGD Motif in SARS-CoV-2 and Potential Inhibitors for Virus Infection. Front Pharmacol. 2020; 11:912. doi:10.3389/fphar.2020.00912.
13. Tresoldi I, Sangiuolo C F, Manzari V, Modesti A. SARS-COV-2 and infectivity: Possible increase in infectivity associated to integrin motif expression. J Med Virol. 2020; 92(10):1741-2. doi:10.1002/jmv.25831.
14. Aguirre C, Meca-Lallana V, Barrios-Blandino A, Del Rio B, Vivancos J. Covid-19 in a patient with multiple sclerosis treated with natalizumab: May the blockade of integrins have a protective role? Mult Scler Relat Disord. 2020; 44:102250. doi:10.1016/j.msard.2020.102250.
15. Lu X, Lu D, Scully M, Kakkar V. The role of integrins in cancer and the development of anti-integrin therapeutic agents for cancer therapy. Perspect Medicin Chem. 2008; 2:57-73.
16. Amruta N, Rahman A A, Pinteaux E, Bix G. Neuroinflammation and fibrosis in stroke: The good, the bad and the ugly. J Neuroimmunol. 2020; 346:577318. doi:10.1016/j.jneuroim.2020.577318.
17. Rahman A A, Amruta N, Pinteaux E, Bix G J. Neurogenesis After Stroke: A Therapeutic Perspective. Transl Stroke Res. 2021; 12(1):1-14. doi:10.1007/s12975 00841-w.
18. Kandikattu H K, Venkateshaiah S U, Kumar S, Mishra A. IL-15 immunotherapy is a viable strategy for COVID-19. Cytokine & growth factor reviews. 2020; 54:24-31.
19. Ishibashi Y, Reiman D A, Nishikawa A. Invasion of human respiratory epithelial cells by Bordetella pertussis: possible role for a filamentous hemagglutinin Arg-Gly-Asp sequence and alpha5beta1 integrin. Microb Pathog. 2001; 30(5):279-88. doi:10.1006/mpat.2001.0432.
20. Feire A L, Koss H, Compton T. Cellular integrins function as entry receptors for human cytomegalovirus via a highly conserved disintegrin-like domain. Proc Natl Acad Sci USA. 2004; 101(43):15470-5. doi:10.1073/pnas.0406821101.
21. Xiao J, Palefsky J M, Herrera R, Berline J, Tugizov S M. The Epstein-Barr virus BMRF-2 protein facilitates virus attachment to oral epithelial cells. Virology. 2008; 370(2):430-42. doi:10.1016/j.virol.2007.09.012.
22. Zarate S, Romero P, Espinosa R, Arias C F, Lopez S. VP7 mediates the interaction of rotaviruses with integrin alphavbeta3 through a novel integrin-binding site. J Virol. 2004; 78(20):10839-47. doi:10.1128/JVI.78.20.10839-10847.2004.
23. Akula S M, Pramod N P, Wang F Z, Chandran B. Integrin alpha3beta1 (CD 49c/29) is a cellular receptor for Kaposi's sarcoma-associated herpesvirus (KSHV/HHV-8) entry into the target cells. Cell. 2002; 108(3):407-19. doi:10.1016/s0092-8674(02)00628-1.
24. Gire S K, Goba A, Andersen K G, Sealfon R S, Park D J, Kanneh L et al. Genomic surveillance elucidates Ebola virus origin and transmission during the 2014 outbreak. Science. 2014; 345(6202):1369-72. doi:10.1126/science.1259657.
25. Clarke N E, Fisher M J, Porter K E, Lambert D W, Turner A J. Angiotensin converting enzyme (ACE) and ACE2 bind integrins and ACE2 regulates integrin signalling. PLoS One. 2012; 7(4):e34747. doi:10.1371/journal.pone.0034747.
26. Chen B, Tian E K, He B, Tian L, Han R, Wang S et al. Overview of lethal human coronaviruses. Signal Transduct Target Ther. 2020; 5(1):89. doi:10.1038/s41392-020-0190-2.
27. Beddingfield B J, Iwanaga N, Chapagain P P, Zheng W, Roy C J, Hu T Y et al. The integrin binding peptide, ATN-161, as a novel therapy for SARS-CoV-2 infection. Basic to Translational Science. 2021; 6(1):1-8.
28. Park E J, Myint P K, Appiah M G, Darkwah S, Caidengbate S, Ito A et al. The Spike Glycoprotein of SARS-CoV-2 Binds to beta1 Integrins Expressed on the Surface of Lung Epithelial Cells. Viruses. 2021; 13(4). doi:10.3390/v13040645.
29. Cianfrocca M, Kimmel K, Gallo J, Cardoso T, Brown M, Hudes G et al. Phase 1 trial of the antiangiogenic peptide ATN-161 (Ac-PHSCN-NH ₂), a beta integrin antagonist, in patients with solid tumours. British journal of cancer. 2006; 94(11):1621-6.
30. Lv X, Li Z, Guan J, Zhang J, Xu B, He W et al. ATN-161 reduces virus proliferation in PHEV-infected mice by inhibiting the integrin alpha5beta1-FAK signaling pathway. Vet Microbiol. 2019; 233:147-53. doi:10.1016/j.vetmic.2019.04.029.
31. Donate F, Parry G C, Shaked Y, Hensley H, Guan X, Beck I et al. Pharmacology of the novel antiangiogenic peptide ATN-161 (Ac-PHSCN-NH₂): observation of a U-shaped dose-response curve in several preclinical models of angiogenesis and tumor growth. Clinical Cancer Research. 2008; 14(7):2137-44.
32. Edwards D N, Salmeron K, Lukins D E, Trout A L, Fraser J F, Bix G J. Integrin alpha5beta1 inhibition by ATN-161 reduces neuroinflammation and is neuroprotective in ischemic stroke. J Cereb Blood Flow Metab. 2020; 40(8):1695-708. doi:10.1177/0271678X19880161.
33. Kliche J, Kuss H, Ali M, Ivarsson Y. Cytoplasmic short linear motifs in ACE2 and integrin beta3 link SARS-CoV-2 host cell receptors to mediators of endocytosis and autophagy. Sci Signal. 2021; 14(665). doi:10.1126/scisignal.abf1117.
34. Meszaros B, Samano-Sanchez H, Alvarado-Valverde J, Calyseva J, Martinez-Perez E, Alves R et al. Short linear motif candidates in the cell entry system used by SARS-CoV-2 and their potential therapeutic implications. Sci Signal. 2021; 14(665). doi:10.1126/scisignal.abd0334.
35. Han K, Blair R V, Iwanaga N, Liu F, Russell-Lodrigue K E, Qin Z et al. Lung Expression of Human Angiotensin-Converting Enzyme 2 Sensitizes the Mouse to SARS-CoV-2 Infection. Am J Respir Cell Mol Biol. 2021; 64(1):79-88. doi:10.1165/rcmb.2020-03540C.
36. Bao L, Deng W, Huang B, Gao H, Liu J, Ren L et al. The pathogenicity of SARS-CoV-2 in hACE2 transgenic mice. Nature. 2020; 583(7818):830-3. doi:10.1038/s41586-020-2312-y.
37. Corman V M, Landt O, Kaiser M, Molenkamp R, Meijer A, Chu D K et al. Detection of 2019 novel coronavirus (2019-nCoV) by real-time R T-PCR. Euro Surveil. 2020; 25(3). doi:10.2807/1560-7917.ES.2020.25.3.2000045.
38. Iwanaga N, Cooper L, Rong L, Beddingfield B, Crabtree J, Tripp R A et al. Novel ACE2-IgG1 fusions with improved in vitro and in vivo activity against SARS-CoV2. bioRxiv. 2020.
39. www.who.int/emergencies/diseases/novel-coronavirus-2019 ACdC-pWHOA.
40. coronavirus.jhu.edu/map.html.
41. Ramanathan M, Ferguson I D, Miao W, Khavari P A. SARS-CoV-2 B.1.1.7 and B.1.351 Spike variants bind human ACE2 with increased affinity. bioRxiv. 2021. doi:10.1101/2021.02.22.432359.
42. Laha S, Chakraborty J, Das S, Manna S K, Biswas S, Chatterjee R. Characterizations of SARS-CoV-2 mutational profile, spike protein stability and viral transmission. Infect Genet Evol. 2020; 85:104445. doi:10.1016/j.meegid.2020.104445.
43. Stewart P L, Nemerow G R. Cell integrins: commonly used receptors for diverse viral pathogens. Trends in microbiology. 2007; 15(11):500-7.
44. Montagutelli X, Prot M, Levillayer L, Salazar E B, Jouvion G, Conquet L et al. The B1. 351 and P. 1 variants extend SARS-CoV-2 host range to mice. bioRxiv. 2021.
45. Golden J W, Cline C R, Zeng X, Garrison A R, Carey B D, Mucker E M et al. Human angiotensin-converting enzyme 2 transgenic mice infected with SARS-CoV-2 develop severe and fatal respiratory disease. JCI Insight. 2020; 5(19). doi:10.1172/jci.insight.142032.
46. Jiang R D, Liu M Q, Chen Y, Shan C, Zhou Y W, Shen X R et al. Pathogenesis of SARS-CoV-2 in Transgenic Mice Expressing Human Angiotensin-Converting Enzyme 2. Cell. 2020; 182(1):50-8 e8. doi:10.1016/j.ce11.2020.05.027.
47. Guan W-j, Ni Z-y, Hu Y, Liang W-h, Ou C-q, He J-x et al. Clinical characteristics of coronavirus disease 2019 in China. New England journal of medicine. 2020; 382(18):1708-20.
48. Huang C, Huang L, Wang Y, Li X, Ren L, Gu X et al. 6-month consequences of COVID-19 in patients discharged from hospital: a cohort study. Lancet. 2021; 397(10270):220-32. doi:10.1016/50140-6736(20)32656-8.
49. Yinda C K, Port J R, Bushmaker T, Owusu T O, Avanzato V A, Fischer R J et al. K18-hACE2 mice develop respiratory disease resembling severe COVID-19. bioRxiv. 2020. doi:10.1101/2020.08.11.246314.
50. Sun S H, Chen Q, Gu H J, Yang G, Wang Y X, Huang X Y et al. A Mouse Model of SARS-CoV-2 Infection and Pathogenesis. Cell Host Microbe. 2020; 28(1):124-33 e4. doi: 10.1016/j.chom.2020.05.020.
51. Winkler E S, Bailey A L, Kafai N M, Nair S, McCune B T, Yu J et al. SARS-CoV-2 infection of human ACE2-transgenic mice causes severe lung inflammation and impaired function. Nat Immunol. 2020; 21(11):1327-35. doi:10.1038/s41590-020-0778-2.
52. Pan F, Ye T, Sun P, Gui S, Liang B, Li L et al. Time course of lung changes at chest C T during recovery from coronavirus disease 2019 (COVID-19). Radiology. 2020; 295(3):715-21.
53. Xu Z, Shi L, Wang Y, Zhang J, Huang L, Zhang C et al. Pathological findings of COVID-19 associated with acute respiratory distress syndrome. Lancet Respir Med. 2020; 8(4):420-2. doi:10.1016/52213-2600(20)30076-X.
54. Tian S, Hu W, Niu L, Liu H, Xu H, Xiao S Y. Pulmonary Pathology of Early-Phase 2019 Novel Coronavirus (COVID-19) Pneumonia in Two Patients With Lung Cancer. J Thorac Oncol. 2020; 15(5):700-4. doi:10.1016/j.jtho.2020.02.010.
55. Schaller T, Hirschbuhl K, Burkhardt K, Braun G, Trepel M, Markl B et al. Postmortem Examination of Patients With COVID-19. JAMA. 2020; 323(24):2518-20. doi:10.1001/jama.2020.8907.
56. Fox S E, Akmatbekov A, Harbert J L, Li G, Quincy Brown J, Vander Heide R S. Pulmonary and cardiac pathology in African American patients with COVID-19: an autopsy series from New Orleans. Lancet Respir Med. 2020; 8(7):681-6. doi:10.1016/S2213-2600(20)30243-5.
57. Ackermann M, Verleden S E, Kuehnel M, Haverich A, Welte T, Laenger F et al. Pulmonary Vascular Endothelialitis, Thrombosis, and Angiogenesis in Covid-19. N Engl J Med. 2020; 383(2):120-8. doi:10.1056/NEJMoa2015432.
58. Magro C, Mulvey J J, Berlin D, Nuovo G, Salvatore S, Harp J et al. Complement associated microvascular injury and thrombosis in the pathogenesis of severe COVID-19 infection: A report of five cases. Transl Res. 2020; 220:1-13. doi:10.1016/j.trs1.2020.04.007.
59. Brumeanu T D, Vir P, Shashikumar S, Karim A F, Kar S, Chung K K et al. A Human-Immune-System mouse model for COVID-19 research (DRAGA mouse: HLA-A2.HLA-DR4.RaglKO.IL-2Rgammac KO.NOD). bioRxiv. 2020. doi:10.1101/2020.08.19.251249.
60. Kliche J, Kuss H, Ali M, Ivarsson Y. Cytoplasmic short linear motifs in ACE2 and integrin 03 link SARS-CoV-2 host cell receptors to mediators of endocytosis and autophagy. Science Signaling. 2021; 14(665).
61. Makowski L, Olson-Sidford W, W-Weisel J. Biological and clinical consequences of integrin binding via a rogue RGD motif in the SARS CoV-2 spike protein. Viruses. 2021; 13(2):146.
62. Calver J, Joseph C, John A, Organ L, Fainberg H, Porte J et al. S31 The novel coronavirus SARS-CoV-2 binds RGD integrins and upregulates avb3 integrins in Covid-19 infected lungs. BMJ Publishing Group Ltd; 2021.
63. Hussein H A, Walker L R, Abdel-Raouf U M, Desouky S A, Montasser A K M, Akula S M. Beyond RGD: virus interactions with integrins. Archives of virology. 2015; 160(11):2669-81.
64. Kandikattu H K, Venkateshaiah S U, Verma A K, Mishra A. Tacrolimus (FK506) treatment protects allergen-, IL-5- and IL-13-induced mucosal eosinophilia. Immunology. 2021
65. Bristow M R, Zisman L S, Altman N L, Gilbert E M, Lowes B D, Minobe W A et al. Dynamic Regulation of SARS-Cov-2 Binding and Cell Entry Mechanisms in Remodeled Human Ventricular Myocardium. JACC Basic Transl Sci. 2020; 5(9):871-83. doi:10.1016/j.jacbts.2020.06.007.
66. Lv X, Li Z, Guan J, Hu S, Zhang J, Lan Y et al. Porcine Hemagglutinating Encephalomyelitis Virus Activation of the Integrin alpha5beta1-FAK-Cofilin Pathway Causes Cytoskeletal Rearrangement To Promote Its Invasion of N2a Cells. J Virol. 2019; 93(5). doi:10.1128/JVI.01736-18.
67. Khalili P, Arakelian A, Chen G, Plunkett M L, Beck I, Parry G C et al. A non-RGD-based integrin binding peptide (ATN-161) blocks breast cancer growth and metastasis in vivo. Molecular cancer therapeutics. 2006; 5(9):2271-80.
68. Cianfrocca M E, Kimmel K A, Gallo J, Cardoso T, Brown M M, Hudes G et al. Phase 1 trial of the antiangiogenic peptide ATN-161 (Ac-PHSCN-N H(2)), a beta integrin antagonist, in patients with solid tumours. Br J Cancer. 2006; 94(11):1621-6. doi:10.1038/sj.bjc.6603171.
69. Sundaram A, Chen C, Khalifeh-Soltani A, Atakilit A, Ren X, Qiu W et al. Targeting integrin alpha5beta1 ameliorates severe airway hyperresponsiveness in experimental asthma. J Clin Invest. 2017; 127(1):365-74. doi:10.1172/JCI88555.
70. Xu J, Tao J, Wang J. Design and Application in Delivery System of Intranasal Antidepressants. Front Bioeng Biotechnol. 2020; 8:626882. doi:10.3389/fbioe.2020.626882.
71. Fortuna A, Alves G, Serralheiro A, Sousa J, Falcao A. Intranasal delivery of systemic-acting drugs: small-molecules and biomacromolecules. Eur J Pharm Biopharm. 2014; 88(1):8-27. doi:10.1016/j.ejpb.2014.03.004.
72. Homayun B, Lin X, Choi H J. Challenges and Recent Progress in Oral Drug Delivery Systems for Biopharmaceuticals. Pharmaceutics. 2019; 11(3). doi:10.3390/pharmaceutics11030129.
73. Wu, F., et al. “A New Coronavirus Associated with Human Respiratory Disease in China.” Nature 579 (2020): 265-269. PubMed: 32015508.
74. Amanat, F., et al. “A Serological Assay to Detect SARSCoV-2 Seroconversion in Humans.” Nat. Med. 26 (2020): 1033-1036. PubMed: 32398876.
75. Beddingfieid, vanaga, N., Chapagain, P.P., Zheng, W., Roy, C.J., Hu, T N., Kolis, J. K. and Bix, G. J., 2021. The integrin binding peptide, ATN-161, as a novel therapy for SARS-CoV-2 infection. Basic to Translational Science, 6(1), pp.1-8.
76. Othman H, Haifa Ben Messaoud, Oussema Khamessi, Hazem Ben Mabrouk, Kais Ghedira, Avani Bharuthram, Florette Treurnicht, Ikechukwu Achilonu, Yasien Sayed, Najet Srairi-Abid. 2021. SARS-CoV-2 spike protein unlikely to bind to integrins via the Arg-Gly-Asp (RGD) motif of the Receptor Binding Domain: evidence from structural analysis and microscale accelerated molecular dynamics. bioRxiv 2021.05.24.445335; doi: https://doi.org/10.1101/2021.05.24.445335
77. Bhattarai N, Baral P. Gerstman B S, Chapagain P P. Structural and Dynamical Differences in the Spike Protein RBD in the SARS-CoV-2 Variants B.1.1.7 and B.1.351. J Phys Chem B. 2021 Jun. 10. doi: 10.1021/acs.jpcb.1c01626. Epub ahead of print. PMID: 34110159.
78. Huang, Y., Yang, C., Xu, Xf. et al. Structural and functional properties of SARS-CoV-2 spike protein: potential antivirus drug development for COVID-19. Acta Parmacol Sin 419 1141-1149 (2020). https://doi.org/10.1.038/s41401-020-0485-4.
79. Doñate F, Parry G C, Shaked Y, Hensley H, Guan X, Beck 1, Tel-Ts Plunkett M L, Nlanuia M, Shaw D E, Kerbel R S, Mazar A P. Pharmacology of the novel antiangiogenic peptide ATN-161 (Ac-PHSCN-NIH): observation of a U-shaped dose-response curve in several preclinical models of angiogenesis and tumor growth. Clin Cancer Res. 2008 Apr. 1; 147):2137-44, doi: 10.1158/1078-0432,CCR-07-4530. PMID: 18381955,
80. Simpson-Haidaris P J, Courtney M A, Wright 1W, Goss R, Harmsen A, Gigliotti F. 1998, Induction of fibrinogen expression in the lung epithelium during Pneumocystis carinii pnewnonia. Infection and Immunity, 1 Sep. 1998, 66(9):4431-4439, DOI: 10.1128/iai.66.9.4431-4439, 1998.

It will be understood that various details of the presently disclosed subject matter can be changed without departing from the scope of the subject matter disclosed herein. Furthermore, the foregoing description is for the purpose of illustration only, and not for the purpose of limitation.

1. A method of treating a human coronavirus (hCoV) infection, comprising administering an effective amount of an integrin inhibitor of an α5- or αv-containing integrin to a subject in need thereof.

2. The method of claim 1, wherein the integrin inhibitor is an α5β1 integrin inhibitor.

3. The method of claim 1, wherein the integrin inhibitor comprises an ATN-161.

4. The method of claim 3, wherein the ATN-161 is provided as pharmaceutically-acceptable salt.

5-6. (canceled)

7. The method of claim 1, and further comprising administering the integrin inhibitor intranasally, pulmonarily, orally, or parenterally.

8. The method of claim 1, wherein the hCoV infection is severe acute respiratory syndrome (SARS) CoV or SARS-CoV-2.

9. The method of claim 1, wherein the hCoV includes a spike protein that binds to an angiotensin-converting enzyme 2 (ACE2) of a target host cell.

10. The method of claim 9, wherein the hCoV binds to the α5- or αv-containing integrin.

11-12. (canceled)

13. The method of claim 1, wherein the integrin inhibitor is administered prophylactically to the subject.

14. The method of claim 13, and further comprising administering the integrin inhibitor to the subject at least three times a week.

15-19. (canceled)

20. The method of claim 18, wherein the subject has at least one symptom of SARS or COVID.

21-23. (canceled)

24. The method of claim 20, and further comprising administering the integrin inhibitor to the subject until the at least one symptom has resolved.

25. The method of claim 1, wherein the method reduces viral activity in the subject.

26. The method of claim 1, wherein the method protects against detrimental histopathological changes.

27. A pharmaceutical composition for use in the treatment of a human coronavirus (hCoV) infection, comprising an integrin inhibitor of an α5- or αv-containing integrin and a pharmaceutically-acceptable carrier.

28. The composition of claim 27, wherein the integrin inhibitor is an α5β1 integrin inhibitor.

29. The composition of claim 27, wherein the integrin inhibitor comprises an ATN-161.

30. The composition of claim 29, wherein the ATN-161 is provided as pharmaceutically-acceptable salt.

31. The composition of claim 30, wherein the ATN-161 is provided as a trifluoroacetate salt

32. The composition of claim 29, where the ATN-161 is adapted for administration intranasally, pulmonarily, orally, or parenterally.

33-58. (canceled)


SEQUENCE LISTING (8 sequences)

SEQ ID NO: 1 - protein, 5 aa, Artificial Sequence
  Synthesized
PHSCN

SEQ ID NO: 2 - DNA, 20 bp, Artificial Sequence
  Primer
gaccccaaaatcagcgaaat

SEQ ID NO: 3 - DNA, 24 bp, Artificial Sequence
  Primer
tctggttactgccagttgaatctg

SEQ ID NO: 4 - DNA, 24 bp, Artificial Sequence
  Probe
accccgcattacgtttggtggacc

SEQ ID NO: 5 - DNA, 23 bp, Artificial Sequence
  Primer
cgatctcttgtagatctgttctc

SEQ ID NO: 6 - DNA, 25 bp, Artificial Sequence
  Probe
taaccagaatggagaacgcagtggg

SEQ ID NO: 7 - DNA, 20 bp, Artificial Sequence
  Primer
ggtgaaccaagacgcagtat

SEQ ID NO: 8 - protein, 9 aa, Artificial Sequence
  Synthesized
CRRETAWAC